(19) United States
(12) Patent Application Publication
(10) Pub. No.: US 2020/0281470 A1
(43) Pub. Date: Sep. 10, 2020

(54) INFORMATION MEASURING DEVICE, COMMUNICATION DEVICE, SYSTEM, METHOD, AND NON-TRANSITORY RECORDING MEDIUM STORING PROGRAM

(71) Applicants: OMRON HEALTHCARE Co., Ltd., Kyoto (JP); OMRON Corporation, Kyoto-shi (JP)

(72) Inventors: Hiroshi NAKAJIMA, Kyoto (JP); Hirotaka WADA, Kyoto (JP); Tomohiro KUKITA, Kyoto (JP); Daisuke NOZAKI, Kyoto (JP); Natsuko HORIGUCHI, Kyoto (JP); Tamio UEDA, Kyoto (JP)

(21) Appl. No.: 16/884,197

(22) Filed: May 27, 2020

Related U.S. Application Data
(63) Continuation of application No. PCT/JP2018/043763, filed on Nov. 28, 2018.

(30) Foreign Application Priority Data
Dec. 1, 2017 (JP) 2017-232059

(51) Int. Cl. A61B 5/00; A61B 5/0205; G06F 21/62; G06F 21/32

(52) U.S. Cl. CPC A61B 5/0026; A61B 5/0022; G06F 21/32; G06F 21/6245; A61B 5/02055

(57) ABSTRACT

The present invention identifies whether or not a user consents to providing personal information including biological information to a third party. A biological information measuring device having a function for communicating with a communication device having a communication area of a specific range and a function for measuring information of a living body pertaining to a user includes a transmission control unit configured to transmit, when the biological information measuring device is detected as having entered the communication area, identification information unique to the biological information measuring device to the communication device, and a reception control unit configured to receive, when the communication device has stored in advance the identification information and personal information of the user associated with the identification information, a response signal from the communication device indicating that consent is regarded as having been obtained for providing, to a third party, the personal information and the information of the living body in association with each other.

### CROSS-REFERENCE TO RELATED APPLICATIONS

This is a continuation of International Application, 2017-232059 with an international filing date of Dec. 1, 2017, and PCT/JP2018/043763, with an international filing date of Nov. 28, 2018, and filed by applicant, the disclosure of which is hereby incorporated by reference in its entirety.

### TECHNICAL FIELD

The present invention relates to a biological information measuring device, a communication device, a system, a method, and a non-transitory recording medium storing a program having, for example, a function for providing personal information including biological information to a third party (hereinafter, also referred to as third-party provision).

### BACKGROUND ART

As the utilization of personal information increases with the development of economic and social computerization, the protection of personal information, including biological information, has become increasingly important in recent years to protect personal rights, including the protection of privacy. On the other hand, diverse utilization of personal information through the employment of information communication technology achieves precise reflection of personal needs in business and provision of rapid services and the like, and is indispensable for both business activities and civilian life. Therefore, ideas regarding the protection and usefulness of personal information need to be fully considered in the actual handling of personal information and, in the protection of personal information, it is required to consider a balance between protection of personal information and appropriate and effective employment.

Personal information is defined as information pertaining to a living individual, including information allowing identification of a specific individual by a name, a date of birth, or other description included in the information (including information that can easily cross-checked with other information, thereby identifying a specific individual), or information including a personal identification reference numeral (refer to, for example, “Guidelines on the Act on the Protection of Personal Information (General Rules)” Personal Information Protection Commission of November 2016 (partially revised March 2017)).

Personal information that includes biological information corresponds to “the results of a medical check-up or other examination for the prevention and early detection of a disease conducted on a principal by a medical doctor or other person engaged in duties related to medicine”, which is a type of sensitive personal information. For acquisition and third-party provision of sensitive personal information, the consent of the individual is required, in principle (note that, “third-party provision by opt-out”, which allows provision of personal data to a third party without obtaining the consent of the individual in advance after meeting prescribed conditions, is not allowed in the case of sensitive personal information). In obtaining consent, the content of a reasonable and appropriate range considered necessary for the individual to make a determination regarding consent must be clearly indicated in accordance with the scale and nature of the business, the handling status of personal data (including the nature and amount of personal data handled), and the like.

Further, the following rules are provided for the “consent of the individual” in the acquisition of sensitive personal information. When a personal information handling operator appropriately acquires sensitive personal information from an individual in writing, orally, or the like, it is understood that, by the individual providing the information, the personal information handling operator has obtained the consent of the individual to acquire the information. Note that a “personal information handling operator” refers to an entity who provides a personal information database or the like for business use. In this context, the term “business” in “provides . . . for business use” refers to the same kind of activities that are repeatedly and continuously performed for a certain purpose, and that is recognized as business by social wisdom, regardless of whether for-profit or not-for-profit. Further, “third party” generally refers to a person other than the individual specified by the personal data and the personal information handling operator intended to provide the personal data, regardless of whether it is a person, a corporation, or another organization.

That is, when personal information including biological information is to be provided to a third party, first, by the individual providing the personal information including the biological information to the personal information handling operator, the individual is regarded as having provided consent for the personal information handling operator to acquire the biological information. Furthermore, to provide personal information including biological information to a third party, it is necessary for the personal information handling operator to clearly indicate to the individual the content of a reasonable and appropriate range considered necessary for the individual to make a determination regarding consent, and then receive the consent of the individual.

There is a technology for providing information to others in which, before a communication terminal is used to initiate data communication for communicating data, which includes the information, between devices, whether or not each device is permitted to be connected in the wireless communication area is authenticated. For example, in JP 2005-229597 A, before a Bluetooth (trade name) type mobile terminal and an appliance are connected by wireless communication and initiate data communication, biological information of the individual is utilized to authenticate whether or not the individual devices are permitted to connect in the wireless communication area.

### SUMMARY OF INVENTION

According to the technology disclosed in JP 2005-229597 A, after authentication, a user in a wireless communication area can transmit personal information including his or her biological information from his or her own device to a desired device of a personal information handling operator and, when the device of this personal information handling operator obtains the personal information including this biological information, the consent of the individual for the acquisition of this personal information may be regarded as received.

Nevertheless, in the technology disclosed in JP 2005-229597 A, it is not possible to clearly indicate to the individual the content of a reasonable and appropriate range considered necessary for the individual to make a determination regarding consent related to the provision of personal information including biological information to a third party. Furthermore, in the technology disclosed in Cited Document 1, there is a problem in that it is not possible to confirm whether or not the individual consents to the content using the device of the other party or the device of the individual.

The present invention has been made with reference to the above circumstances, and an object thereof is to provide a biological information measuring device, a communication device, a system, a method, and a non-transitory recording medium storing a program capable of identifying whether or not a user consents to third-party provision of personal information including biological information.

In order to solve the problems described above, the present invention adopts the following configuration.

That is, a biological information measuring device according to an aspect of the present invention is a biological information measuring device having a function for communicating with a communication device having a communication area of a specific range and a function for measuring information of a living body pertaining to a user, the biological information measuring device including a transmission control unit configured to transmit, when the biological information measuring device is detected as having entered the communication area, identification information unique to the biological information measuring device to the communication device, and a reception control unit configured to receive, when the communication device has stored in advance the identification information and personal information of the user associated with the identification information, a response signal from the communication device indicating that consent is regarded as having been obtained for providing, to a third party, the personal information and the information of the living body in association with each other.

In the configuration described above, when consent is obtained to provide, to a third party, the information of the living body (hereinafter also simply referred to as “biological information”) and the personal information in association with each other, and the biological information measuring device enters the communication area of the communication device, the identification information is transmitted to this communication device and, when this identification information (received by the communication device) exists in the identification information of the biological information measuring device stored by the communication device, provision of the biological information in association with this identification information to a third party is regarded as permitted. This allows the communication device to provide, to a third party, the biological information and the personal information of the user in association with each other.

Note that the consent for providing, to a third party, the biological information and the personal information in association with each other refers to, for example, storing the identification information of the biological information measuring device used by the user in the communication device in advance using some technique, and further regarding the consent described above as established when the biological information measuring device has entered the communication area.

In the biological information measuring device according to the aspect described above, the biological information measuring device further includes a presentation control unit configured to present, to the user, consent determination information indicating content necessary for the user to make a determination whether or not to consent to providing, to a third party, the information of the living body and personal information of the user corresponding to the living body in association with each other, and a determination reception control unit configured to receive, from the user, a signal indicating a determination result made by the user regarding consent on the basis of the content indicated by the consent determination information. The transmission control unit is configured to transmit the identification information and the information of the living body to the communication device when the signal indicating the determination result indicates consent and the biological information measuring device is detected as having entered the communication area.

In the configuration described above, the content necessary for determining whether or not to consent to providing, to a third party, the biological information and the personal information in association with each other is presented to the user, allowing the user to check the necessary content for his or her consent. Further, information indicating whether or not the user has consented to providing, to a third party, the biological information and the personal information in association with each other on the basis of this necessary content can be received. Furthermore, when consent for providing the information to a third party has been received and the biological information measuring device enters the communication area of the communication device, the identification information and the biological information are transmitted to this communication device and, when this identification information (received by the communication device) exists in the identification information of the biological information measuring device stored by the communication device, provision of the biological information in association with this identification information to a third party is regarded as allowed. This allows the communication device to provide the biological information and the personal information of the user in association with each other to a third party.

In the biological information measuring device according to the aspect described above, the presentation control unit is configured to present the consent determination information to the user when the biological information measuring device is detected as having entered the communication area, and the determination reception control unit is configured to receive a signal indicating the determination when the biological information measuring device is detected as having entered the communication area.

In the configuration described above, when the biological information measuring device is detected as having entered the communication area of the communication device, the user is presented with an inquiry for consent to provide, to a third party, the biological information and the personal information in association with each other. With the user being requested to provide consent upon entry into the communication area of the communication device, the communication device can, for example, change the content for each communication area of the communication device as appropriate, deliver the content to the biological information measuring device, and present the content to the user. Further, the signal indicating the determination made by the user is received in the communication area, and thus a signal indicating the determination made can be immediately transmitted to the communication device, making it possible to provide a biological information measuring device in which the communication device can immediately provide, to a third party, the biological information and the personal information in association with each other.

In the biological information measuring device according to the aspect described above, the presentation control unit is configured to present the consent determination information to the user without the biological information measuring device detected as having entered the communication area, and the determination reception control unit is configured to receive a signal indicating the determination without the biological information measuring device detected as having entered the communication area.

In the configuration described above, the user is presented with content necessary for determining whether or not to consent to providing, to a third party, biological information and personal information without the biological information measuring device detected as having entered the communication area of the communication device (before entry). Thus, the user can determine whether or not to consent to providing the information to a third-party before entry into the communication area. Further, because the biological information measuring device can receive a signal indicating the determination made by the user before entry into the communication area, as long as the user makes a determination before entering the communication area, the user no longer needs to make a determination of consent after entering the communication area. As a result, in the communication area, there is time to engage in work other than this determination. Furthermore, because the transmission control unit of the biological information measurement device can immediately transmit the unique identification information and the biological information to the communication device when the biological information measuring device enters the communication area, the communication device can provide, to a third party, the biological information and the personal information in association with each other at substantially the same time as entry into the communication area.

In the biological information measuring device according to the aspect described above, the biological information measuring device further includes a storage unit configured to store in advance personal information and a password of a user, in association with each other, for performing encrypted communication, an authentication control unit configured to transmit the personal information and the password to the communication device and perform authentication for performing encrypted communication with the communication device, and a communication control unit configured to initiate encrypted communication with the communication device when the authentication is completed.

In the configuration described above, encrypted communication between the biological information measuring device and the communication device can be performed. Accordingly, it is possible to provide a biological information measuring device capable of performing secure communication with not only unique identification information but also a password associated with the personal information of the user.

In a communication device according to the aspect described above, the communication device is configured to communicate with a biological information measuring device configured to measure information of a living body pertaining to a user and has a communication area in a specific range. The communication device includes a reception unit configured to receive identification information unique to the biological information measuring device and the information of the living body measured by the biological information measuring device from the biological information measuring device when the user consents to providing, to a third party, the information of the living body and personal information of the user in association with each other and the biological information measuring device has entered the communication area, a storage unit configured to store in advance identification information of at least one biological information measuring device and personal information of a user using the biological information measuring device in association with each other, and a consent control unit configured to regard that the user corresponding to personal information associated with the identification information had consented to providing, to a third party, the personal information and the information of the living body in association with each other when the identification information being received is stored in the storage unit.

In the configuration described above, when the user consents to providing, to a third party, the biological information and the personal information in association with each other, and the biological information measuring device enters the communication area, the communication device can receive the identification information of the biological information measuring device and the biological information and acquire identification information and biological information as a set.

Then, it is possible to provide a communication device capable of deciding whether or not identification information of the acquired set of information is already stored in the storage unit and, when this identification information is stored, providing, to a third party, biological information and personal information in association with each other.

In a communication device according to the aspect described above, the communication device is configured to communicate with a biological information measuring device configured to measure information of a living body pertaining to a user and having a communication area in a specific range. The communication device includes a reception unit configured to receive identification information unique to the biological information measuring device and the information of the living body from the biological information measuring device when the biological information measuring device has entered the communication area, a transmission control unit configured to transmit, to the biological information measuring device, consent determination information indicating content necessary for a user corresponding to the personal information to determine whether or not to consent to providing, to a third party, the information of the living body in association with personal information associated with the identification information, and a consent control unit configured to regard the user as having consented to providing, to a third party, the personal information and the information of the living body in association with each other when a signal indicating that the user consented on the basis of the content indicated by the consent determination information is received from the biological information measuring device.

In the configuration described above, when the biological information measuring device enters the communication area, the communication device receives the identification information of the biological information measuring device and the biological information of the user. Then, the communication device can transmit, to the biological information measuring device, consent determination information indicating the content necessary for the user corresponding to the personal information associated with the identification information to determine whether or not to consent to providing the information to a third party.

Accordingly, it is possible to provide a communication device capable of regarding consent for providing biological information to a third party as having been obtained when a signal indicating such consent for providing biological information to a third party is received from a biological information measuring device.

In the communication device according to the aspect described above, the communication device further includes a storage unit configured to store in advance authentication information of a user for performing encrypted communication, an authentication control unit configured to transmit the authentication information to the biological information measuring device and perform authentication for performing encrypted communication with the biological information measuring device, and a communication control unit configured to initiate encrypted communication with the biological information measuring device when the authentication is completed.

In the configuration described above, encrypted communication between the biological information measuring device and the communication device can be performed. Accordingly, it is possible to provide a communication device capable of performing secure communication with not only unique identification information but also a password associated with the personal information of the user.

In the communication device according to the aspect described above, the communication device further includes an identification information decision control unit configured to decide whether or not identification information obtained when the biological information measuring device logs into the communication device using the authentication information differs from the identification information associated with the personal information, and a notification control unit configured to notify a user corresponding to the personal information when decision with the identification information obtained differing from the identification information associated with the personal information is made.

In the configuration described above, when the identification information stored in advance in association with the personal information in the communication device differs from the identification information of the device logged into by a password, the user included in the personal information is notified that the login is not from the regular biological information measuring device (informed by, for example, email, a social networking service (SNS), or the like). Accordingly, it is possible to provide a communication device capable of preventing another person from impersonating the user without the knowledge of the user.

In the communication device according to the aspect described above, the communication device further includes an identification information decision control unit configured to decide whether or not login information obtained when the biological information measuring device logs into the communication device using the authentication information differs from login information of the past included in login history, and a notification control unit configured to notify, when decision with the login information obtained differing from the login information exists in the login history is made, information indicating the decision to a user corresponding to the personal information.

In the configuration described above, it is decided whether or not the login information included in the history of logins and the like recorded in the communication device differs from the identification information of the device logged into using the authentication information. When the login information of the device logged into differs from the login information recorded in the history, the regular user of this biological information measuring device stored by the communication device can be notified that the login is not from the regular biological information measuring device. Accordingly, it is possible to provide a communication device capable of preventing another person from impersonating the user without the knowledge of the user.

In a system according to the aspect described above, the system includes a biological information measuring device configured to measure information of a living body pertaining to a user, and a communication device having a communication area of a specific range. The biological information measuring device includes a presentation control unit configured to present, to the user, consent determination information indicating content necessary for the user to determine whether or not to consent to providing, to a third party, the information of the living body and personal information of the user in association with each other, a determination reception control unit configured to receive, from the user, a signal indicating a determination result made by the user regarding consent on the basis of the content indicated by the consent determination information, and a transmission control unit configured to transmit identification information unique to the biological information measuring device and the information of the living body to the communication device when the signal indicating the determination result indicates consent and the biological information measuring device has entered the communication area. The communication device includes a storage unit configured to store in advance identification information of at least one biological information measuring device and personal information of a user using the biological information measuring device in association with each other, a decision control unit configured to decide whether or not the identification information transmitted by the biological information measuring device is stored in the storage unit, and a consent control unit configured to regard the user as having consented to providing, to a third party, personal information associated with the identification information and the information of the living body in association with each other when the identification information transmitted by the biological information measuring device is decided to be stored in the storage unit.

In the configuration described above, consent determination information indicating the content necessary for determining whether to provide, to a third party, the biological information and the personal information in association with each other is presented to the user, allowing the user to obtain the necessary content for his or her consent. Further, the biological information measuring device can receive information indicating whether the user has consented to providing, to a third party, the biological information and the personal information in association with each other on the basis of the consent determination information indicating this necessary content. Further, when the user has consented to providing the information to a third party and the biological information measuring device has entered the communication area of the communication device, the identification information and the biological information are transmitted to this communication device. Then, when this identification information (received by the communication device) exists in the identification information of a blood pressure measurement device stored by the communication device, provision of the biological information in association with this identification information to a third party is regarded as allowed. Furthermore, the communication device can notify the biological information measuring device of the content necessary for the user corresponding to the personal information associated with the identification information to determine whether or not to consent to providing the information to a third party. Accordingly, the communication device can regard consent for providing biological information to a third party as having been obtained when a signal indicating such consent is received from the biological information measuring device.

In the biological information measuring device according to the aspect described above, the biological information measuring device further includes a measurement control unit configured to measure the information of the living body when the biological information measurement device is detected as having entered the communication area.

In the configuration described above, once the biological information measurement device enters the communication area, the information of the living body is measured in that area. Thus, the biological information is measured in a specific communication area. In this case, the biological information measuring device can acquire biological information in a specific communication area. As a result, the biological information measuring device can collect biological information in a specific communication area.

In the biological information measuring device according to the aspect described above, the biological information measuring device further includes a measurement control unit configured to measure the information of the living body without the biological information measuring device detected as having entered the communication area.

In the configuration described above, biological information is measured before the biological information measuring device enters the communication area, and thus the biological information can be transmitted from the biological information measuring device to the communication device or the like immediately after entry into the communication area. Further, the biological information measuring device can measure biological information in a desired environment, making it possible to acquire more beneficial biological information.

In the biological information measuring device according to the aspect described above, the identification information unique to the biological information measuring device includes a media access control (MAC) address.

In the configuration described above, because the biological information measuring device includes a MAC address, it is not necessary to newly generate identification information, and information unique to the manufactured product can be utilized as the identification information.

In the biological information measuring device according to the aspect described above, the identification information unique to the biological information measuring device corresponds to the personal information of the user.

In the configuration described above, because the identification information of the biological information measuring device corresponds to the personal information of the user, the user can be associated with the biological information measuring device on a per biological information measuring device basis.

In the biological information measuring device according to the aspect described above, the biological information measuring device further includes a decision control unit configured to decide whether the identification information unique to the biological information measuring device corresponds to the personal information of the user.

In the configuration described above, it is possible to decide whether the identification information included in the biological information measuring device corresponds to the personal information of the user and, when corresponding, associate the user with the biological information measuring device on a per biological information measuring device basis and, when not corresponding, recognize a need to acquire the personal information of the user using a different technique.

In the biological information measuring device according to the aspect described above, the determination reception control unit is configured to operate by a touch of a button of hardware or software.

In the configuration described above, the determination reception control unit is set as a physically present hardware button or software button and, by being touched by the user, can receive a determination indicating that the information may be provided to a third party.

In the biological information measuring device according to the aspect described above, the biological information measuring device and the communication device communicate using a short-range wireless communication system.

In the configuration described above, the biological information measuring device and the communication device communicate using a short-range wireless communication system, and thus, for example, Bluetooth (trade name) is used. Other communication methods also exist, such as Transfer Jet (trade name), Zigbee (trade name), and IrDA (trade name), and these may be used as well.

In the biological information measuring device according to the aspect described the above, the biological information measuring device includes a mobile information terminal and a biological information measuring instrument. The mobile information terminal includes the presentation control unit, the determination reception control unit, the transmission control unit, and the reception control unit. The biological information measuring instrument includes a sensor configured to measure the information of the living body, and a transmission control unit configured to transmit the information of the living body to the mobile information terminal.

In the configuration described above, the mobile information terminal is, for example, a smartphone, and the biological information measuring instrument is, for example, a blood pressure monitor having a transmission function. With the mobile information terminal thus including a main control unit, the biological information measuring instrument need only include a function for measuring biological information and a function for transmitting biological information to the mobile information terminal. As a result, the main control is performed by the mobile information terminal, making it possible to use an existing biological information measuring instrument (an existing blood pressure monitor, for example).

In the biological information measuring device according to the aspect described above, the consent control unit includes a decision control unit configured to decide whether or not the identification information received is stored in the storage unit, and an authentication control unit that is configured to regard the user corresponding to personal information associated with the identification information as having consented to providing, to a third party, the personal information and the information of the living body in association with each other when the identification information received is decided to be stored in the storage unit.

In the above-described configuration, the consent control unit stores, in the storage unit in advance, identification information corresponding to a user who consented to provide, to a third party, the personal information and the biological information in association with each other, and decides whether or not the identification information received is stored in this storage unit. When the received identification information has been stored in advance, the user is regarded as having consented to providing, to a third party, the personal information and the biological information in association with each other. Thus, when consent to providing, to a third party, the personal information and biological information in association with each other is stored, it is possible to provide, to a third party, the personal information and the biological information in association with each other immediately when the corresponding identification information is received.

In the biological information measuring device according to the aspect described above, the biological information measuring device further includes a change control unit configured to connect to a different communication device or change the third party when the user does not consent to the necessary content.

In the configuration described above, it is possible to provide a biological information measuring device capable of changing to a different communication device or to a different third party when the user does not consent to the necessary content, and thus provide the expectation of an increased likelihood of discovering a communication device or a third party with which the user will consent to the necessary content.

According to the present invention, it is possible to provide a biological information measuring device, a communication device, a system, a method, and a non-transitory recording medium storing a program capable of identifying whether or not a user consents to third-party provision of personal information including biological information.

### BRIEF DESCRIPTION OF DRAWINGS

FIG. 1 is a drawing schematically illustrating an example of an application scenario of a system including a blood pressure measurement device and a communication device according to an embodiment.

FIG. 2 is a drawing schematically illustrating an example of a hardware configuration of the blood pressure measurement device of the embodiment.

FIG. 3 is a drawing illustrating an example of an appearance of the blood pressure measurement device of FIG. 2.

FIG. 4 is a drawing schematically illustrating an example of a hardware configuration of the communication device according to the embodiment.

FIG. 5 is a drawing schematically illustrating an example of a software configuration of the blood pressure measurement device of the embodiment.

FIG. 6A is a drawing schematically illustrating an example of a software configuration of the communication device of the embodiment.

FIG. 6B is a drawing illustrating an example of a table stored by a storage unit of the communication device according to the embodiment.

FIG. 7 is a flowchart illustrating an example of a processing procedure of the blood pressure measurement device according to the embodiment.

FIG. 8 is a flowchart illustrating an example of a processing procedure of step S701 of FIG. 7.

FIG. 9 is a flowchart illustrating an example of a processing procedure of the communication device according to the embodiment.

FIG. 10 is a drawing schematically illustrating an example of a hardware configuration of a blood pressure measurement device according to a modified example.

### DESCRIPTION OF EMBODIMENTS

An embodiment according to an aspect of the present invention (hereinafter, also referred to as “the present embodiment”) will be described below with reference to the drawings. Note that, in the following embodiment, parts denoted by the same reference numbers perform the same operations, and a repetitive explanation will be omitted.

### Application Example

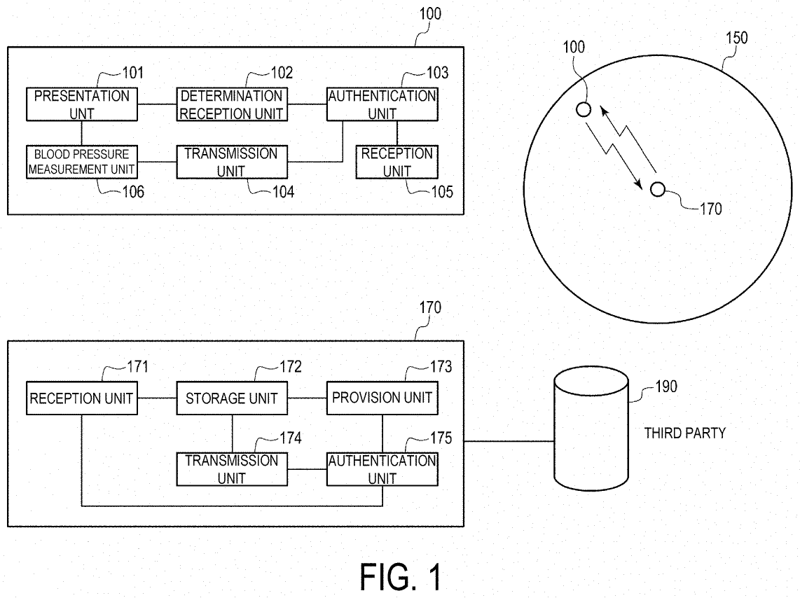

First, with reference to FIG. 1, an example of a scenario in which the present invention is applied will be described. FIG. 1 schematically illustrates an example of an application scenario of a blood pressure measurement device 100 and a communication device 170 according to the present embodiment. In the blood pressure measurement device 100 (corresponding to the “biological information measuring device” of the present invention) according to the present embodiment, a presentation unit 101 presents, to the user, consent determination information indicating content necessary for determining whether or not to consent to providing, to a third party, biological information and personal information of the user in association with each other. When a determination reception unit 102 receives the determination made by the user in accordance with this content, receives a signal indicating a determination that the information may be provided to a third party, and detects entry into a communication area of the communication device, a transmission unit 104 transmits identification information of the blood pressure measurement device 100 and the biological information to the communication device.

In the communication device 170 according to the present embodiment, a reception unit 171 receives the identification information transmitted from the transmission unit 104 and, when the identification information and the corresponding personal information are stored in a storage unit 172, a provision unit 173 regards the user as having consented to providing the information to a third party, and provides the biological information and the personal information of the user in association with each other to a server 190 of the third party.

Further, an authentication unit 103 of the blood pressure measurement device 100 and an authentication unit 175 of the communication device 170 make it possible to perform encrypted communication between the blood pressure measurement device 100 and the communication device 170.

Note that, in the present embodiment, personal information is defined as information pertaining to a living individual, including information allowing identification of a specific individual by a name, a date of birth, or other description included in the information (including information that can easily cross-checked with other information, thereby identifying a specific individual), or information including a personal identification reference numeral. Further, in the present embodiment, for convenience, biological information may be treated as information different from personal information. For example, in the case of the description “biological information and personal information in association with each other,” it is implied that this personal information does not include biological information.

As illustrated in FIG. 1, the biological information measuring device 100 is configured to communicate with a communication device having a communication area of a specific range, is attached to a living body, and measures a blood pressure of the living body (an example of “information of a living body” of the present invention), and includes the presentation unit 101 configured to present, to the user, consent determination information indicating content necessary for determining whether or not to consent to providing, to a third party, the information of the living body and personal information of the user corresponding to the living body in association with each other, the determination reception unit 102 configured to receive, from the user, a signal indicating a determination result made by the user regarding whether the information of the living body and personal information of the user corresponding to the living body may be provided to a third party in association with each other, the transmission unit 174 configured to transmit, when a signal indicating a determination result that the information may be provided to a third party is received and the biological information measuring device 100 has entered the communication area, identification information unique to the biological information measuring device and the information of the living body to the communication device, and a reception unit 105 configured to receive, when the communication device has stored in advance the identification information and personal information of the user associated with the identification information, a response signal from the communication device indicating that consent is regarded as having been obtained for providing, to a third party, the personal information and the information of the living body in association with each other.

Note that the presentation unit 101 corresponds to the “presentation control unit” of the present invention, the determination reception unit 102 corresponds to the “determination reception control unit” of the present invention, the transmission unit 104 corresponds to the “transmission control unit” of the present invention, and the reception unit 105 corresponds to the “reception control unit” of the present invention.

A blood pressure measurement unit 106, as long as a device is configured to perform blood pressure measurement, may use any blood pressure measurement method. The blood pressure measurement unit 106 measures blood pressure by, for example, an oscillometric method. Further, the blood pressure measurement device 100 may measure the pulse, for example, without measuring blood pressure (or in addition to measuring blood pressure). That is, the blood pressure measurement device 100 of the present embodiment may measure anything as long as the information is biological information. Further, the blood pressure measurement device 100 may be configured such that components other than the blood pressure measurement unit 106 are constituted as a separate device. That is, the presentation unit 101, the determination reception unit 102, the authentication unit 103, the transmission unit 104, and the reception unit 105 may be included in a separate device such as, for example, a mobile information terminal such as a smartphone, a mobile phone, or a mobile personal computer. In this case, the blood pressure measurement unit 106 is a blood pressure instrument, measures the blood pressure of a living body, and transmits the measured blood pressure data to the separate device described above. The blood pressure instrument and the separate device communicate using, for example, a short-range wireless communication system (Bluetooth, for example). Further, this communication between the blood pressure instrument and the separate device may also be realized in a wired manner rather than wirelessly (for example, one-to-one communication using a universal serial bus (USB) cable or the like).

When the communication device 170 has a communication area of a specific range, presents to the user the consent determination information indicating content necessary for determining whether or not to consent to providing, to a third party, personal information and biological information of the user in association with each other, and receives a signal indicating that the user has determined that the personal information and the biological information may be provided to the third party, the user is regarded as having consented to providing, to the third party, the personal information and the biological information in association with each other when the blood pressure measurement device 100 enters this communication area and the identification information unique to the blood pressure measurement device 100 is stored in the communication device 170.

As described above, in the present embodiment, in the blood pressure measurement device 100, the presentation unit 101 presents, to the user, consent determination information indicating content necessary for determining whether or not to consent to providing, to a third party, the personal information and the biological information of the user in association with each other. When the determination reception unit 102 of the blood pressure measurement device 100 receives a signal indicating a determination that the user consents to providing these information to a third party and the blood pressure measurement device 100 has entered the communication area of the communication device 170, the transmission unit 104 transmits the identification information and the biological information to the communication device 170. In the communication device 170, when the storage unit 172 has stored this identification information and the personal information associated with this information in advance, the provision unit 173 regards the user as having consented to providing, to a third party, the personal information and the biological information in association with each other. Thus, according to the present embodiment, upon presenting, to the user, consent determination information indicating the content necessary for determining whether or not to consent to providing, to a third party, the personal information in association, it is possible to obtain consent from the user. As a result, the communication device can provide, to a third party, biological information and identification information of a user acquired in advance, in association with each other.

### Configuration Example

### Hardware Configuration

### Blood Pressure Measurement Device

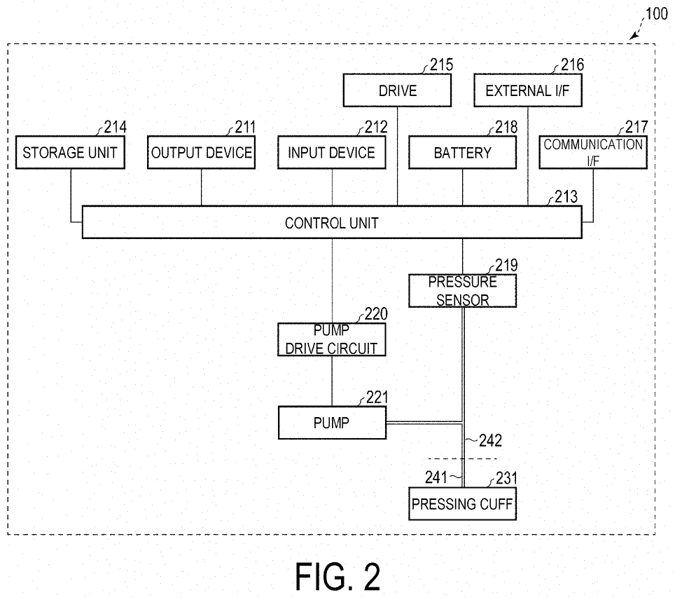

Next, an example of a hardware configuration of the blood pressure measurement device 100 according to the present embodiment will be described using FIG. 2.

As illustrated in FIG. 2, the blood pressure measurement device 100 according to the present embodiment includes an output device 211, an input device 212, a control unit 213, a storage unit 214, a drive 215, an external interface 216, a communication interface 217, and a computer to which a battery 218 is electrically connected. The blood pressure measurement device 100 further includes a pressure sensor 219, a pump drive circuit 220, a pump 221, and a pressing cuff 231. The blood pressure measurement device 100 according to the present embodiment corresponds to the “biological information measuring device” of the present invention. Note that, in FIG. 2, the communication interface and the external interface are written as “communication I/F” and “external I/F”, respectively.

The control unit 213 includes a central processing unit (CPU), a random-access memory (RAM), a read-only memory (ROM), and the like, and controls each component in accordance with information processing. The storage unit 214 is, for example, an auxiliary storage device such as a hard disk drive or a solid state drive, and stores a third-party provision and consent program and a blood pressure measurement program executed by the control unit 213 and/or blood pressure measurement data, which is data of blood pressure measured by the blood pressure measurement device.

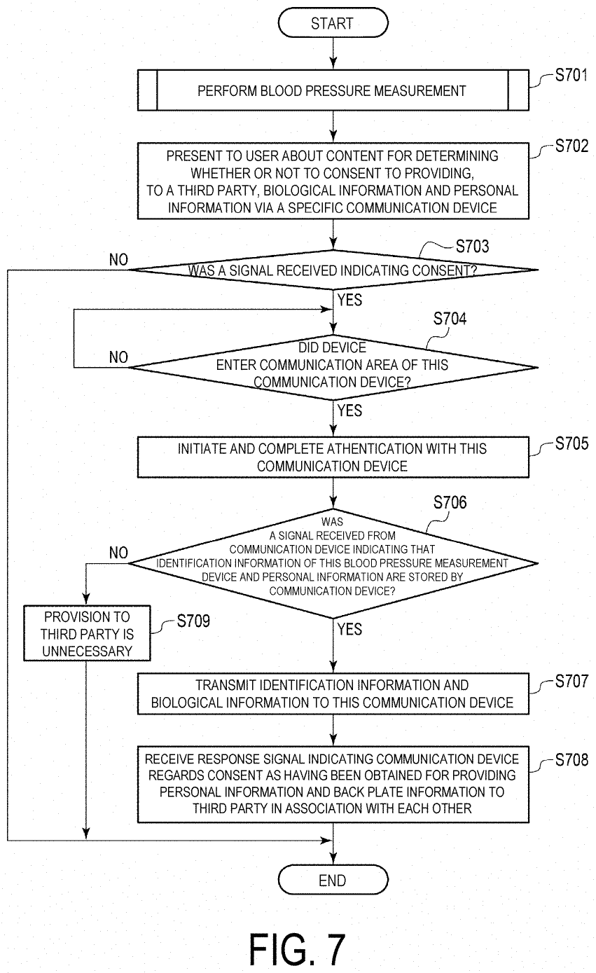

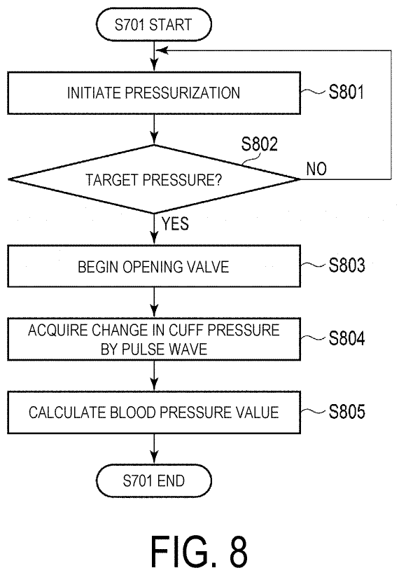

The third-party provision and consent program is a program for executing processing (FIG. 7) for providing the user with consent determination information indicating content for determining whether or not to consent to providing, to a third party, biological information measured by the blood pressure measurement device 100 and personal information via a specific communication device and, when the user consents, transmitting the identification information of the blood pressure measurement device 100 to the communication device 170 described above, concluding whether or not to execute third-party provision on the basis of the identification information and, upon concluding that third-party provision is to be executed, notifying the blood pressure measurement device 100 of execution. Further, the blood pressure measurement program is a program for executing processing (FIG. 8) for utilizing the pressing cuff 231 to measure the blood pressure of a user having the cuff attached. Furthermore, the blood pressure measurement data is time series data of blood pressure obtained by executing the blood pressure measurement program. Details will be described later.

The communication interface 217 is, for example, a short-range wireless communication system (for example, Bluetooth) module, a wireless local area network (LAN; for example, a Wi-Fi (trade name) module, or the like, and is an interface configured to perform wireless communication via a network. The communication interface 217 is an interface configured to connect the blood pressure measurement device 100 with the communication device 170. The communication interface 217 is controlled by the control unit 213. The communication interface 217 relays information from the communication device 170 received via the network to the control unit 213. Communication via the network employs a wireless system. Note that the communication interface 217 may be capable of transmitting information to the communication device 170 via the network. The network may be another type of network, such as a LAN in a hospital.

As another example, the communication interface 217 may be connected to the communication device 170 via a separate device such as, for example, a mobile information terminal such as a smart phone, a mobile phone, or a mobile personal computer. In this case, the blood pressure measurement device 100 may transmit the blood pressure measurement, time series data of a blood pressure value, and the like to the mobile information terminal, and the mobile information terminal may perform all processing other than blood pressure measurement (processing other than S701 in FIG. 7).

The input device 212 is a device configured to perform input, such as a mouse or a keyboard, for example. The output device 211 is a device configured to perform output, such as a display or a speaker, for example. The external interface 216 is a USB port or the like, and is an interface configured to connect to an external device, such as the pressure sensor 219 and/or the pump drive circuit 220, for example.

The storage unit 214 is a medium that stores information such as a recorded program by electrical, magnetic, optical, mechanical, or chemical action so that a computer or other device, a machine, or the like can read information such as the program. The blood pressure measurement device 100 may obtain the third-party provision and consent program, the blood pressure measurement program, and/or the blood pressure measurement data from this storage unit 214.

The drive 215 is, for example, a compact disc (CD) drive, a digital versatile disc (DVD) drive, or the like, and is a device configured to read a program stored in a storage medium. The type of the drive 215 may be selected as appropriate according to the type of the storage medium. The third-party provision and consent program, the blood pressure measurement program, and/or the blood pressure measurement data may be stored in this storage medium. Here, as an example of a storage medium, a storage medium of a disk type, such as a CD or a DVD, is illustrated. Nevertheless, the type of storage medium is not necessarily limited to a disk type, and may be a type other than a disk type. Examples of a storage medium other than a disk type include a semiconductor memory such as flash memory.

The battery 218 is, for example, a rechargeable secondary battery. The battery 218 supplies power to each element mounted to the body of blood pressure measurement device 100. The battery 218 supplies power to, for example, the output device 211, the input device 212, the control unit 213, the storage unit 214, the drive 215, the external interface 216, the communication interface 217, the pressure sensor 219, the pump drive circuit 220, and the pump 221.

The pressure sensor 219 is, for example, a piezoresistive pressure sensor. The pressure sensor 219 detects pressure in the pressing cuff 231 through a flexible tube 241 and a first flow path formation member 242 constituting a first flow path. The pressure sensor 219 outputs pressure data (for example, time series data of a pressure value) to the control unit 213.

The pump drive circuit 220 drives or brakes the pump 221 (turns the pump 221 on or off, for example) on the basis of a control signal from the control unit 213.

The pump drive circuit 220, when a decision is made to inject a fluid, drives the pump 221 that injects the fluid into the pressing cuff 231.

The pump 221 is a piezoelectric pump, for example. The pump 221 is connected to the pressing cuff 231 in a way that allows fluid to flow to the pressing cuff 231 through the first channel. The pump 221 can supply a fluid (air, for example) through the first flow path to the pressing cuff 231. Note that the pump 221 is equipped with an exhaust valve (not illustrated) controlled to open or close as the pump 221 is turned on or off. That is, this exhaust valve closes when the pump 221 is turned on, encapsulating air within the pressing cuff 231. On the other hand, this exhaust valve opens when the pump 221 is turned off, discharging the air in the pressing cuff 231 through the first flow path and into the atmosphere. Note that, this exhaust valve has the function of a check valve, preventing the discharged air from flowing in reverse.

Further, different from this, the control unit 213 may control the on/off state of the pump 221 and control the opening and closing of the exhaust valve separately.

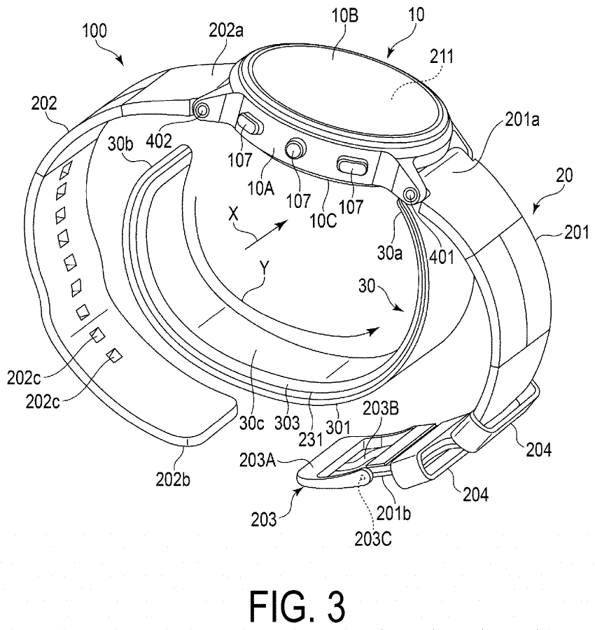

The pressing cuff 231 will be described later with reference to FIG. 3.

Further, the amount of fluid injected by the pump 221 into the pressing cuff 231 may be fixed. The pump 221 supplies a fixed flow at 10 standard cubic centimeter per minute (sccm), for example. The sccm is regulated by atmospheric pressure and temperature (for example, regulated at 1 atmospheric pressure and 0 degrees Celsius). In this case, as long as the elapsed time since the start of inflow is ascertained, the amount of fluid injected into the pressing cuff 231 can be calculated. In the present embodiment, the amount of fluid injected into the pressing cuff 231 can be calculated by measuring the elapsed time since the control unit 213 turned on the pump drive circuit 220.

Further, a data table indicating a relationship between drive voltage and flow rate of the pump drive circuit 220 is stored in advance in the storage unit 214, and the control unit 213 may obtain the flow rate from the drive voltage of the pump drive circuit 220 by replacing a voltage value with a flow rate with reference to the data table.

Note that, with respect to the specific hardware configuration of the blood pressure measurement device 100, components can be omitted, substituted, and added as appropriate in accordance with the embodiment. For example, the control unit 213 may include a plurality of processors. The blood pressure measurement device 100 may be constituted by a plurality of information processing devices. Further, the blood pressure measurement device 100 may use a general-purpose desktop personal computer (PC), a tablet PC, or the like in addition to an information processing device dedicatedly designed for the provided service.

### Structure of Blood Pressure Measurement Device

Next, an example of a structure of the blood pressure measurement device 100 will be described with reference to FIG. 3.

FIG. 3 is a drawing illustrating an example of an appearance of a blood pressure measurement device according to this invention.

The blood pressure measurement device 100 is, for example, a wrist-watch type wearable device. The blood pressure measurement device 100 includes a body 10, a belt 20, and a cuff structure 30.

The body 10 is configured to allowing mounting of a plurality of elements, such as elements of a control system of the blood pressure measurement device 100.

The body 10 includes a case 10A, a glass 10B, and a rear lid 10C.

The case 10A has, for example, a substantially short cylindrical shape. The case 10A includes a pair of protruding lugs configured to attach the belt 20, in two locations on a side surface thereof.

The glass 10B is attached to an upper portion of the case 10A. The glass 10B has, for example, a circular shape.

The rear lid 10C is removably attached to a lower portion of the case 10A so as to face the glass 10B.

The body 10 includes the output device 211 and an operation unit 107.

The output device 211 displays various information. The output device 211 is provided inside the body 10 at a position visible by the subject through the glass 10B. The output device 211 is a liquid crystal display (LCD). The output device 211 may be an organic electroluminescence (EL) display. The output device 211 may be provided with a function for displaying various information, but is not limited thereto. The output device 211 may include a light emitting diode (LED).

The operation unit 107 is included in the input device 212, and is an element configured to input various commands to the blood pressure measurement device 100. The operation unit 107 is provided on a side surface of the body 10. The operation unit 107 includes one or more push type switches, for example. The operation unit 107 may be a pressure sensitive type (resistance type) or a proximity type (capacitance type) touch panel type switch. The operation unit 107 may be provided with a function for inputting various instructions to the blood pressure measurement device 100, but is not limited thereto.

Here, an example of a switch included in the operation unit 107 will be described.

The operation unit 107 includes a measurement switch configured to provide a start or stop instruction to the blood pressure measurement. Further, the operation unit 107 may include a home switch configured to return the display screen of the output device 211 to a predetermined home screen, and a record calling switch configured to cause the output device 211 to display a measurement record such as a past blood pressure or activity level.

Note that, on the body 10 are mounted a plurality of elements other than the output device 211 and the operation unit 107. The plurality of elements provided to the body 10 are described later.

The belt 20 is configured to be capable of being wrapped around a target measurement site (for example, a left wrist, a right wrist) of a subject (user). A width direction of the belt 20 is an X direction. On the other hand, a direction in which the belt 20 is wrapped around the target measurement site is a Y direction.

The belt 20 includes a first belt portion 201, a second belt portion 202, a buckle 203, and a belt holding portion 204.

The first belt portion 201 is a band-like object extending from the body 10 to one side (right side in FIG. 3) in one direction. A base portion 201a of the first belt portion 201 near the body 10 is rotatably attached to a pair of lugs of the body 10 by interposing a connecting rod 401.

The second belt portion 202 is a band-like object extending from the body 10 to the other side (left side in FIG. 3) in one direction. The base portion 202a of the second belt portion 202 near the body 10 is rotatably attached to a pair of lugs of the body 10 by interposing a connecting rod 402. A plurality of small holes 202c are formed through the second belt portion 202 in a thickness direction between the base portion 202a of the second belt portion 202 and a tip end portion 202b away from the body 10.

The buckle 203 is configured to be capable of fastening the first belt portion 201 and the second belt portion 202. The buckle 203 is attached to a tip end portion 201b of the first belt portion 201 away from the body 10. The buckle 203 includes a frame body 203A, a buckle tongue 203B, and a connecting rod 203C.

The frame body 203A and the buckle tongue 203B are rotatably attached to the tip end portion 201b of the first belt portion 201 by interposing the connecting rod 203C. The frame body 203A and the buckle tongue 203B are formed from a metal material, for example. The frame body 203A and the buckle tongue 203B may be formed from a plastic material. When the first belt portion 201 and the second belt portion 202 are fastened, the tip end portion 202b of the second belt portion 202 is passed through the frame member 203A. The buckle tongue 203B is inserted through any one of the plurality of small holes 202c of the second belt portion 202.

The belt holding portion 204 is attached between the base portion 201a and the tip end portion 201b of the first belt portion 201. When the first belt portion 201 and the second belt portion 202 are fastened, the tip end portion 202b of the second belt portion 202 is passed through the belt holding portion 204.

### Structure of Cuff

The configuration of the cuff structure 30 will now be described.

The cuff structure 30 is configured to be capable of compressing the target measurement site during blood pressure measurement.

The cuff structure 30 is band-like structure extending in the Y direction. The cuff structure 30 faces an inner peripheral surface of the belt 20. A first end 30a of the cuff structure 30 is attached to the body 10. A second end 30b of the cuff structure 30 is a free end. Thus, the cuff structure 30 is freely separable from the inner peripheral surface of the belt 20.

The cuff structure 30 includes a curler 301, the pressing cuff 231, and a back plate 303.

The curler 301 is disposed at an outermost periphery of the cuff structure 30. In the natural state, the curler 301 is curved in the Y direction. The curler 301 is a resin plate having a predetermined flexibility and hardness. The resin plate is made of polypropylene, for example.

The pressing cuff 231 is disposed along an inner peripheral surface of the curler 301. The pressing cuff 231 is bag-like. The flexible tube 241 (FIG. 2) is attached to the pressing cuff 231. The flexible tube 241 is an element configured to supply a fluid from the body 10 side or discharge a fluid from the pressing cuff 231. The fluid is, for example, air. When the fluid is supplied to the pressing cuff 231, the pressing cuff 231 expands, compressing the target measurement site.

Note that the pressing cuff 231 may include two fluid bags layered in the thickness direction, for example. Each fluid bag is constituted of a stretchable polyurethane sheet, for example. When the fluid is supplied to the pressing cuff 231, the fluid flows into each fluid bag. The expansion of each fluid bag expands the pressing cuff 231.

The back plate 303 is disposed along an inner peripheral surface of the pressing cuff 231. The back plate 303 is band-like. The back plate 303 is constituted of, for example, a resin. The resin is, for example, polypropylene. The back plate 303 functions as a reinforcing plate.

An inner peripheral surface and an outer peripheral surface of the back plate 303 are provided with a plurality of parallel grooves spaced apart from each other in the Y direction and having a V-shaped or U-shaped cross section extending in the X direction. With the back plate 303 prone to bending, the back plate 303 does not prevent the cuff structure 30 from attempting to bend.

### Communication Device

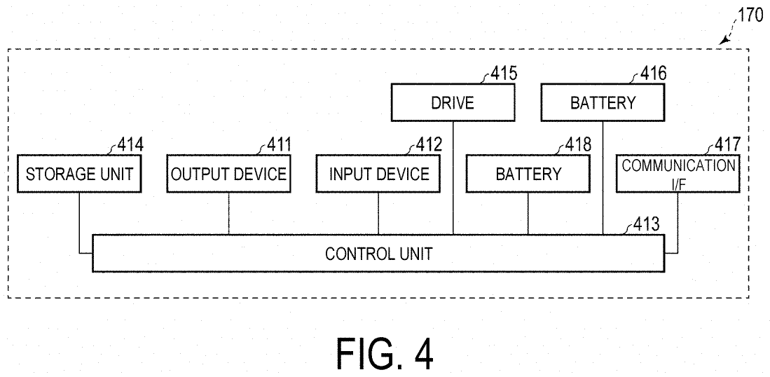

Next, an example of a hardware configuration of the communication device 170 according to the present embodiment will be described using FIG. 4.

As illustrated in FIG. 4, the communication device 170 according to the present embodiment includes an output device 411, an input device 412, a control unit 413, a storage unit 414, a drive 415, an external interface 416, a communication interface 417, and a computer to which a battery 418 is electrically connected. The communication device 170 according to the present embodiment corresponds to the “communication device” of the present invention. Note that, in FIG. 4, the communication interface and the external interface are written as “communication I/F” and “external I/F”, respectively.

The control unit 413 includes a CPU, a RAM, a ROM, and the like, and controls each component in accordance with information processing. The storage unit 414 is, for example, an auxiliary storage device such as a hard disk drive, a solid state drive, or the like, and stores a third-party provision confirmation program executed by the control unit 413, and/or transmission/reception data, which is data transmitted and/or received by the communication device, or the like.

The third-party provision confirmation program is a program for executing processing for storing in advance personal information and identification information of a user who has approved the provision of biological information and personal information to a third party and, when entry into the communication area of the communication device is detected, decides whether the identification information from the blood pressure measurement device 100 is stored and, when this identification information is stored, regards consent as having been established for providing, to a third party, the biological information and personal information of the user corresponding to the living body in association with each other, and transmits a response signal indicating the consent to the blood pressure measurement device 100.

The communication interface 417 is, for example, a short-range wireless communication system (for example, Bluetooth) module, a wireless LAN (for example, Wi-Fi) module, or the like, and is an interface configured to perform wireless communication via the network. The communication interface 417 is an interface configured to connect the communication device 170 to the blood pressure measurement device 100. The communication interface 417 is controlled by the control unit 413. The communication interface 417 relays information from the blood pressure measurement device 100 received via the network to the control unit 413. Communication via the network employs a wireless system. Note that the communication interface 417 may be capable of transmitting information to the communication device 170 via the network. The network may be another type of network, such as a LAN in a hospital.

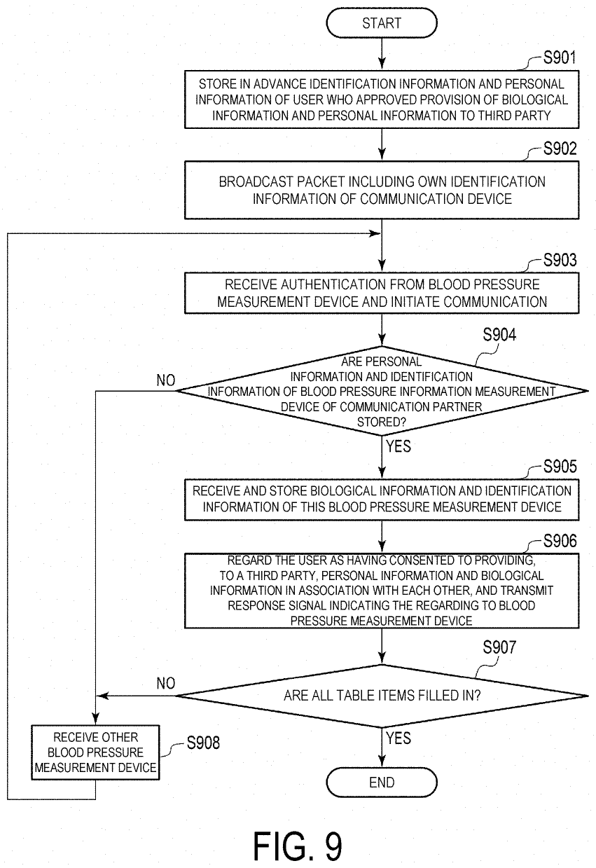

As another example, the communication interface 417 may be connected to the blood pressure measurement device 100 through a separate device such as, for example, a mobile information terminal such as a smart phone, a mobile phone, or a mobile personal computer. In this case, the communication device 170 may broadcast a packet including the identification information of the communication device and transmit the data and the like to the mobile information terminal, and the mobile information terminal may store authentication processing (processing of S903 in FIG. 9) as well as the identification information of the blood pressure measurement device 100 and personal information, and communicate with the communication device 170.

The input device 412 is a device configured to perform input, such as a mouse or a keyboard, for example. The output device 411 is a device configured to perform output, such as a display or a speaker, for example. The external interface 416 is a USB port or the like, and is an interface configured to connect to an external device.

The storage unit 414 is a medium that stores information such as a recorded program by electrical, magnetic, optical, mechanical, or chemical action so that a computer or other device, a machine, or the like can read information such as the program. The communication device 170 may obtain the third-party provision confirmation program and/or transmission/reception data from this storage unit 414.

The drive 415 is, for example, a CD drive, a DVD drive, or the like, and is a device configured to read a program stored in a storage medium. The type of the drive 415 may be selected as appropriate according to the type of the storage medium. The third-party provision confirmation program and/or the transmission/reception data may be stored in this storage medium. Here, as an example of a storage medium, a storage medium of a disk type, such as a CD or a DVD, is illustrated. Nevertheless, the type of storage medium is not necessarily limited to a disk type, and may be a type other than a disk type. Examples of a storage medium other than a disk type include a semiconductor memory such as flash memory.

The battery 418 is, for example, a rechargeable secondary battery. The battery 418 supplies power to each element mounted to the body of blood pressure measurement device 100. The battery 418 supplies power to, for example, the output device 411, the input device 412, the control unit 413, the storage unit 414, the drive 415, the external interface 416, and the communication interface 417.

### Software Configuration

### Blood Pressure Measurement Device

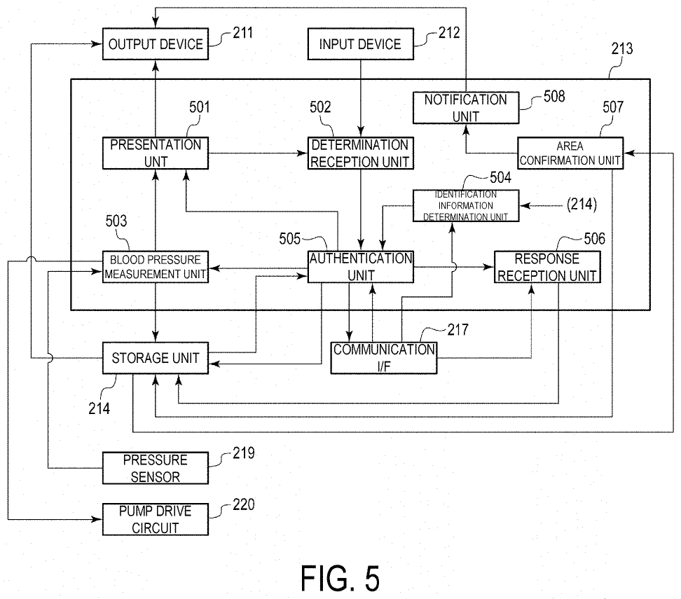

Next, an example of a software configuration of the blood pressure measurement device 100 according to the present embodiment will be described using FIG. 5.

The control unit 213 of the blood pressure measurement device 100 expands the third-party provision and consent program and/or the blood pressure measurement program stored in the storage unit 214 in the RAM when a necessary program is to be executed. Then, the control unit 213 interprets and executes the third-party provision and consent program and/or the blood pressure measurement program expanded in the RAM by the CPU to control each component. Accordingly, as illustrated in FIG. 5, the blood pressure measurement device 100 according to the present embodiment functions as a computer including a presentation unit 501, a determination reception unit 502, a blood pressure measurement unit 503, an identification information decision unit 504, an authentication unit 505, a response reception unit 506, an area confirmation unit 507, and a notification unit 508.

The presentation unit 501 presents, to the user, consent determination information indicating content necessary for determining whether or not to consent to providing, to a third party, biological information of the user and personal information of the user in association with each other. This content is stored in advance in the storage unit 214, for example. Further, the presentation unit 501 may store this content. The presentation unit 501 may present this content to the user when the area confirmation unit 507 confirms that the blood pressure measurement device 100 was detected as having entered the communication area of the communication device 170. Further, the presentation unit 501 may present this content to the user before the area confirmation unit 507 confirms that the blood pressure measurement device 100 was detected as having entered the communication area of the communication device 170. In another scenario, the user may request presentation of the content through the input device 212 without the presentation unit 501 depending on the positional relationship with the communication area of the communication device 170.

The determination reception unit 502 receives a signal from the user indicating the determination made by the user on the basis of the consent determination information presented by the presentation unit 501. The user, for example, delivers a signal indicating the determination made by the user using the input device 212 to the determination reception unit 502. The determination reception unit 502 may be configured to receive the determination made by the user when the area confirmation unit 507 confirms that the blood pressure measurement device 100 has entered the desired communication area of the communication device 170. In this case, the user can make the determination upon confirmation of entry into the communication area of the desired communication device 170, making it possible to observe in substantially real-time the device to which the personal information and the biological information of the user are transmitted.

Apart from this, the determination reception unit 502 may be configured to receive a determination made by the user before the blood pressure measurement device 100 enters the desired communication area of the communication device 170 (that is, without having detected entry into the communication area). With the determination made by the user thus received before entry into the communication area, the number of steps in the processing after the blood pressure measurement device enters the communication area is lower than when the determination is received after entry into the communication area. Accordingly, it is more likely that the time until the biological information of the user and the personal information of the user are provided to a third party in association with each other by the communication device 170 will be quicker than when the determination is received after entry into the communication area.

In the blood pressure measurement unit 503, when the area confirmation unit 507 confirms entry into the communication area of the communication device 170 via the communication interface 217 and authentication is completed with the communication device 170 via the authentication unit 505, the pump drive circuit 220 applies pressure to the cuff structure 30 to initiate blood pressure measurement of the user, and receives the pressure value of the cuff structure 30 measured by the pressure sensor 219. Accordingly, this biological information is measured in a specific communication area. In this case, biological information in a specific communication area can be acquired. As a result, the communication device 170 that receives this biological information can collect biological information in a specific communication area.

Further, the blood pressure measurement unit 503, as long as a device configured to perform blood pressure measurement, may use any blood pressure measurement method. The blood pressure measurement unit 503 measures the blood pressure by, for example, an oscillometric method. The blood pressure value (systolic blood pressure value and diastolic blood pressure value) measured by the blood pressure measurement unit 503 is stored in the storage unit 214. Although not illustrated, the blood pressure measurement device 100 may include a clock to obtain the measured time along with the blood pressure value. The storage unit 214 stores time series data of the blood pressure value, for example. Furthermore, the blood pressure measurement device 100 may include a device configured to obtain position information, and the position at which the blood pressure value is measured may also be stored in the storage unit 214 in association with the blood pressure value. The position information may be acquired by, for example, the blood pressure measurement device 100 including a global positioning system (GPS) receiver.

Further, the blood pressure measurement unit 503 may initiate blood pressure measurement by an instruction from the input device 212 (that is, on the basis of an instruction from the user). For example, before the blood pressure measurement device 100 enters the range of the communication area of the communication device 170, the blood pressure measurement may be initiated blood pressure measurement by an instruction from the user. In this case, at the same time as when the blood pressure measurement device 100 enters this communication area, the time series data of the blood pressure value can be immediately transmitted to the communication device 170 via the storage 214, the authentication unit 505, and the communication interface 217.

The identification information decision unit 504 decides whether the communication device 170 has stored in advance the identification information unique to the blood pressure measurement device 100 and personal information associated with this identification information. For example, the identification information decision unit 504 makes this decision when the communication device 170 is confirmed as being the desired device. The unique identification information is identification information unique to a device such as the blood pressure measurement device 100, and is, for example, a MAC address. The identification information decision unit 504 transmits the identification information of the blood pressure measurement device 100 and the personal information associated with the identification information to the desired communication device 170 via the communication interface 217. Then, the identification information decision unit 504 receives a signal from the communication device 170 via the communication interface 217 indicating whether or not this identification information and the personal information associated with this identification information are stored in the communication device 170. This signal makes it possible for the identification information decision unit 504 to decide whether the communication device 170 has stored the identification information of the blood pressure measurement device 100 and the personal information associated with this identification information. If the communication device 170 has transmitted a signal indicating that the identification information of the blood pressure measurement device 100 or the personal information has not been stored, the identification information decision unit 504 decides that it is not necessary to permit the communication device 170 to provide the biological information and the personal information to a third party. Note that the transmission of the unique identification information and the biological information to the communication device 170 is preferably performed after encrypted communication between the blood pressure measurement device 100 and the communication device 170 has been established.

The authentication unit 505 transmits authentication information including personal information (login name, for example) and the password of the user to the communication device 170, and performs authentication for performing encrypted communication between the blood pressure measurement device 100 and the communication device 170. When this authentication is completed, the blood pressure measurement device 100 initiates encrypted communication with the communication device 170 via the communication interface 217 by using, for example, a cryptographic key corresponding to the authentication information. Note that the storage unit 214 stores in advance the personal information and the password of the user in association with each other to generate, for example, a cryptographic key corresponding to the authentication information. Further, when the blood pressure measurement device 100 transmits and receives data to and from the communication device 170, the data is transmitted and received via the authentication unit 505. Note that this authentication processing may be executed at any time as long as the blood pressure measurement device 100 obtains the identification information of the communication device 170 and the communication device 170 is the desired device. Alternatively, either the communication device 170 or the blood pressure measurement device 100 may generate a cryptographic key, input authentication information, connect to the partner device, and pass the information to the partner. In addition, more simply, a common cryptographic key may be stored in advance in both the blood pressure measurement device 100 and the communication device 170.

The blood pressure measurement device 100 acquires a packet broadcast from the communication device 170, and the area confirmation unit 507 decides whether or not the identification information included in the packet matches any one or more pieces of identification information stored in the storage unit 214 in advance. When the area confirmation unit 507 confirms that matching identification information exists in the storage unit 214, the area confirmation unit approves the communication device 170 as the desired communication partner.

The notification unit 508 receives information indicating that the area confirmation unit 507 approved the communication device 170 as the desired communication partner, and notifies the user via the output device 211 that the blood pressure measurement device 100 has entered the communication area of the communication device 170, which is the desired communication partner.

When the communication device 170 has stored in advance the identification information of the blood pressure measurement device 100 and personal information associated with this identification information, the response reception unit 506 receives a response signal from the communication device 170 via the communication interface 217 indicating that consent is regarded as having been obtained for providing, to a third party, the personal information and the biological information in association with each other.

### Communication Device

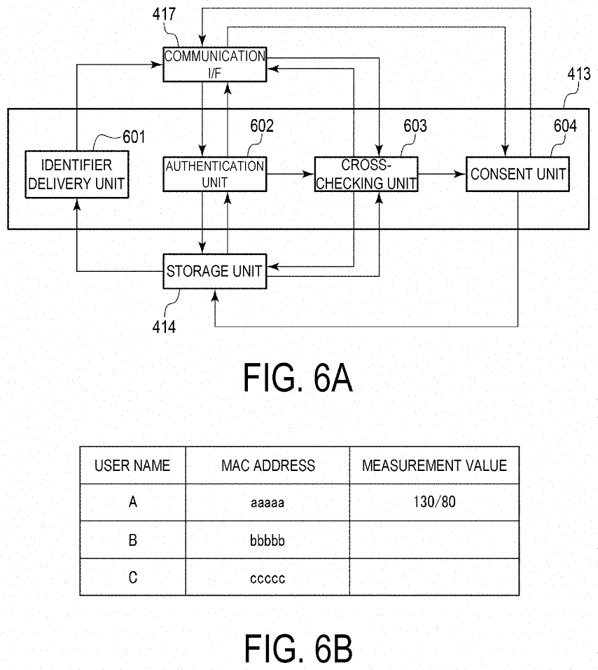

Next, an example of a software configuration of the communication device 170 according to the present embodiment will be described using FIGS. 6A and 6B.

The control unit 413 of the communication device 170 expands the third-party provision confirmation program stored in the storage unit 414 into the RAM when the necessary program is to be executed. Then, the control unit 413 interprets and executes the third-party provision confirmation program expanded into the RAM by the CPU to control each component. As a result, as illustrated in FIG. 6A, the communication device 170 according to the present embodiment functions as a computer including an identifier delivery unit 601, an authentication unit 602, a cross-checking unit 603, and a consent unit 604.

The identifier delivery unit 601 delivers identification information unique to the communication device 170. As a result, the signal (packet) including the identification information of the communication device 170 is broadcast within the communication area.

The authentication unit 602 receives the authentication information including the personal information and the password from the authentication unit 505 of the blood pressure measurement device 100 and, when the authentication information matches the personal information and the password stored in advance, subsequent communication is performed between the blood pressure measurement device 100 and the communication device 170 using a common cryptographic key.

Further, the authentication unit 602 may also transmit the personal information and the password to the blood pressure measurement device 100 to perform authentication encrypted communication with the blood pressure measurement device 100. That is, the authentication unit 602 decides whether the authentication information matches the personal information and the password stored in advance and, when authentication is completed (when the authentication information match the personal information and the password), initiates encrypted communication with the blood pressure measurement device 100 using, for example, a cryptographic key corresponding to the authentication information. Note that, to generate a cryptographic key, the storage unit 414 stores in advance the personal information and the password of the user in association with each other, for example.

On the other hand, the authentication unit 602 may be configured to communicate with the blood pressure measurement device 100 without exchanging the personal information and the password and without encrypting.

The cross-checking unit 603 receives the identification information of the blood pressure measurement device 100 serving as the communication partner and personal information associated with this identification information, and decides whether or not this identification information and personal information are stored in the storage unit 414. When it is decided that the information is stored in the storage unit 414, the communication device 170 transmits a signal to the blood pressure measurement device 100 indicating that the information is stored in the storage unit 414. Upon receiving this signal, the blood pressure measurement device 100 transmits the identification information of the blood pressure measurement device 100 and biological information associated with this identification information to the communication device 170, and the communication device 170 obtains these information. When it is decided that the information is not stored in the storage unit 414, the cross-checking unit 603 decides that the blood pressure measurement device 100 has not obtained advance consent from the user, and transitions to a state of receiving identification information and personal information from another blood pressure measurement device.

Further, the cross-checking unit 603 cross-checks and decides whether or not the identification information obtained when the blood pressure measurement device 100 logged into the communication device 170 using personal information and a password differs from the identification information associated with the personal information. When it is decided that the information differs from the identification information associated with the personal information, the cross-checking unit 603 notifies the user corresponding to the personal information accordingly. When the identification information obtained when the blood pressure measurement device 100 logged into the communication device 170 using personal information and a password differs from the identification information associated with the personal information, it is decided that the user has logged into the communication device 170 by impersonating the original user, and a notification is transmitted to the user corresponding to the personal information (corresponding to the original user) that another person has fraudulently logged in. The original user can receive this notification and perform a procedure such as changing the personal information and/or password of the user, and prevent fraudulent login. Furthermore, when the identification information obtained when the user logged into the communication device 170 differs from the identification information associated with the personal information, the cross-checking unit 603 may issue a notification indicating the fraudulent login and forcibly log out the fraudulent user based on the fraudulent login.

Further, the cross-checking unit 603 may cross-check and decide whether or not the login information obtained when the blood pressure measurement device 100 logged into the communication device 170 using personal information and the password differs from the login information in the history. When it is decided that the information differs from the login information in the login history, the cross-checking unit 603 notifies the user corresponding to the personal information accordingly. In this case as well, the original user can receive this notification and perform a procedure such as changing the personal information and/or password of the user, and prevent fraudulent login. Furthermore, when the login information obtained when the user logged into the communication device 170 differs from the login information in the login history, the cross-checking unit 603 may issue a notification indicating the fraudulent login and forcibly log out the fraudulent user based on the fraudulent login.

When the identification information and personal information received by the blood pressure measurement device 100 are stored in the storage unit 414, the consent unit 604 regards the user corresponding to the personal information associated with the identification information as having consented to providing, to a third party, the personal information and the biological information in association with each other. With, for example, the identification information (here, the MAC address) received from the blood pressure measurement device 100 being stored in the storage unit 414, the consent unit 604 regards the user corresponding to the personal information associated with this MAC address as having consented to providing, to a third party, the personal information and the biological information in association with each other.

Note that, as shown in FIG. 6B, the storage unit 414 records the user name, the MAC address of the blood pressure measurement device 100 owned by the user, and the measured value of the blood pressure in association with each other. In addition to these, information associated with the measured value of the blood pressure (for example, a measured date and time and/or location) may also be recorded. According to FIG. 6B, for example, the user name “A” owns the blood pressure measurement device 100 having a MAC address of “aaaaa”, a systolic blood pressure value and a diastolic blood pressure value of 130 mmHg and 80 mmHg, respectively, are indicated as previous measurements.

Further, according to FIG. 6B, the user names “B” and “C” each have a blank “Measured value” item, indicating that the respective blood pressure values have not yet been measured.

Further, the storage unit 414 stores login information obtained when the blood pressure measurement device 100 logs into the communication device 170. Furthermore, the storage unit 414 stores the identification information of the blood pressure measurement device 100 and the personal information and the password of the user.

### Other

The functions of the blood pressure measurement device 100 and the communication device 170 are described in detail in the operation example described later. Note that, in the present embodiment, an example is described in which each function of the blood pressure measurement device 100 and the communication device 170 are realized by a general-purpose CPU. Nevertheless, a portion or all of the above functions may be realized by one or a plurality of dedicated processors. Further, the functions of the functional configuration of the biological information measuring device 100 and the communication device 170 may be omitted, substituted, and added as appropriate in accordance with the embodiment.

### Operational Example

### Blood Pressure Measurement Device

Next, an operation example of the blood pressure measurement device 100 will be described using FIG. 7 and FIG. 8. FIG. 7 and FIG. 8 are flowcharts illustrating an example of a processing procedure of the blood pressure measurement device 100. Note that the processing procedure described below is merely an example, and each process may be changed to the extent possible. Further, in the processing procedure described below, steps can be omitted, substituted, and added in accordance with the embodiment as appropriate.

### Activation

First, the user activates the blood pressure measurement device 100, causing the blood pressure measurement device 100 thus activated to execute the third-party provision and consent program. The control unit 213 of the blood pressure measurement device 100, in accordance with the processing procedure below, presents to the user consent determination information indicating content for determining whether or not to consent to, providing to a third party, biological information measured by the blood pressure measurement device 100 and personal information via a specific communication device and, when the user consents, transmits the identification information of the blood pressure measurement device 100 to the communication device 170 described above, and concludes whether or not to execute third-party provision on the basis of the identification information. Then, the control unit 213, upon concluding that third-party provision is to be executed, notifies the blood pressure measurement device 100 of execution.

### Step S701

In step S701, the control unit 213 functions as the blood pressure measurement unit 503, and uses the pressure sensor 219, the pump drive circuit 220, and the like to measure the biological information (blood pressure value and/or heart rate, and the like, for example) of the user. The storage unit 214 stores information associated with the measured value of the biological information (the measured date and time and/or location, for example) along with the measured biological information (blood pressure value (systolic blood pressure value and diastolic blood pressure value) and/or heart rate, and the like, for example). Details of the blood pressure measurement performed by the blood pressure measurement device 100 will be described later with reference to FIG. 8.

Note that the blood pressure measurement may be performed at a time desired by the user, may be performed after the blood pressure measurement device 100 has entered (has been detected as having entered) the communication area of the communication device 170, or the like.

### Step S702

In step S702, the control unit 213 functions as the presentation unit 501 and presents to the user content for determining whether to consent to providing the biological information and personal information to a third party via a specific communication device (in the present embodiment, the communication device 170).

### Step S703

In step S703, the control unit 213 functions as the determination reception unit 502, and receives a signal indicating whether or not the user consented to the content presented in step S702 via the input device 212. When the determination reception unit 502 receives a signal indicating that the user consented, the flow proceeds to the next step S704. On the other hand, when the determination reception unit 502 receives a signal indicating that the user does not consent, the subsequent processing is canceled and the program is terminated.

### Step S704

In step S704, the control unit 213 functions as the area confirmation unit 507, and decides whether or not the blood pressure measurement device 100 has entered the communication area of the desired communication device 170. Whether or not the blood pressure measurement device 100 has entered the communication area of the desired communication device 170 is decided by, for example, the blood pressure measurement device 100 receiving the identification information of the communication device 170 broadcast by the communication device 170 and, when this received identification information is stored in the storage unit 214, deciding that the blood pressure measurement device 100 has entered the communication area of the desired communication device 170. Note that the area confirmation unit 507 repeats the decision until entry into the desired communication area.

### Step S705

In step S705, the control unit 213 functions as the authentication unit 505, performs authentication for the blood pressure measurement device 100 to perform encrypted communication with the communication device 170, and completes this authentication. Accordingly, subsequent encrypted communication between the blood pressure measurement device 100 and the communication device 170 is realized.

### Step S706

In step S706, the control unit 213 functions as the identification information decision unit 504, and decides whether or not the communication device 170 has stored in advance the identification information unique to the blood pressure measurement device 100 and personal information associated with this identification information. Specifically, the identification information of the blood pressure measurement device 100 and the personal information associated with this identification information are transmitted to the communication device 170 via the communication interface 217. Then, the identification information decision unit 504 receives a signal from the communication device 170 via the communication interface 217 indicating whether or not this identification information and the personal information associated with this identification information are stored in the communication device 170. The identification information decision unit 504 decides whether or not the identification information of the blood pressure measurement device 100 and the personal information associated with this identification information are stored in the communication device 170 on the basis of this signal.

### Step S707

In step S707, the control unit 213 functions as the blood pressure measurement unit 503 and the authentication unit 505, and transmits the identification information of the blood pressure measurement device 100 and the biological information to the communication device 170. Preferably, this identification information and biological information is transmitted to the communication device 170 upon being encrypted.

### Step S708

In step S708, the control unit 213 functions as the response reception unit 506 and, when the communication device 170 has stored in advance the identification information of the blood pressure measurement device 100 and the personal information associated with the identification information, receives a response signal via the communication interface 217 indicating that consent is regarded as having been obtained for providing, to a third party, the personal information and the biological information in association with each other.

### Step S709

In step S709, the control unit 213 functions as the identification information decision unit 504 and, when the identification information decision unit 504 decides that the communication device 170 does not store the identification information or the personal information of the blood pressure measurement device 100, the control unit 213 decides that provision to a third party is unnecessary, and terminates this program.

Next, a description is given of the blood pressure measurement device 100 measuring the blood pressure value of the user when blood pressure measurement is performed in step S701 of FIG. 7, with reference to FIG. 8. The blood pressure measurement device 100 measures the blood pressure value of the user on the basis of an oscillometric method by the control unit 213 according to the flow chart of FIG. 8, for example.

### Activation

In step S701 in FIG. 7, the blood pressure measurement device 100 executes the blood pressure measurement program.

The control unit 213 of the blood pressure measurement device 100 measures the blood pressure value of the user according to the following processing procedure. While blood pressure measurement by the oscillometric method is described here, another method may be used as long as the blood pressure value of the user is measured.

### Step S801

In step S801, the control unit 213 functions as the blood pressure measurement unit 503, delivers an instruction for the blood pressure measurement unit 503 to perform pressurization via the pump drive circuit 220, and instructs pressurization of the cuff structure 30 (that is, injection of the fluid into the cuff structure 30) using the pump 221, initiating pressurization.

In terms of hardware, at the start of the blood pressure measurement, the control unit 213 initializes the processing memory region of the RAM and outputs a control signal to the pump drive circuit 220. The pump drive circuit 220 opens the exhaust valve of the pump 221 to exhaust the air in the cuff pressing cuff 231 on the basis of the control signal. Subsequently, the control unit 213 performs control for adjusting the pressure sensor 219 to 0 mmHg. Then, the pump drive circuit 220 closes the exhaust valve of the pump 221 and subsequently the pump drive circuit 220 drives the pump 221 to control the injection of the fluid into the cuff structure 30. As a result, the cuff structure 30 is pressurized to the same pressure and expand.

### Step S802

In step S802, the control unit 213 functions as the blood pressure measurement unit 503, the pressure sensor 219 detects the pressure of the cuff structure 30, and the blood pressure measurement unit 503 decides whether or not the pressure has reached a pressure value serving as a preset target. When the pressure value serving as the target has not been reached, the flow returns to step S801, and when the pressure value serving as the target has been reached, the flow proceeds to the next step. Here, the pressure value serving as the target is a pressure value sufficiently higher than the systolic blood pressure value of the user (the systolic blood pressure value+30 mmHg, for example) and is stored in advance in the storage unit 214, or the control unit 213 estimates and concludes the systolic blood pressure value by a predetermined calculation formula during the pressurization of the cuff structure 30.

### Step S803

In step S803, the control unit 213 functions as the blood pressure measurement unit 503 and, when the pressure of the cuff structure 30 is pressurized and reaches the pressure value serving as the preset target, the control unit 213 stops the pump 221 via the pump drive circuit 220, and subsequently performs control so that the exhaust valve of the pump 221 gradually opens. This causes the pressing cuff 231 to contract and gradually depressurize.

### Step S804

In step S804, the control unit 213 functions as the blood pressure measurement unit 503 and, in the depressurization process started from step S803, the pressure sensor 219 detects the pressure value of the cuff structure 30 and outputs a cuff pressure signal. Further, for a depressurization rate, the de-pressurization rate serving as the target is set during pressurization of the cuff structure 30, and the blood pressure measurement part 503 controls the degree of opening of the exhaust valve of the pump 221 so as to achieve the target depressurization rate.

### Step S805

In step S805, the control unit 213 functions as the blood pressure measurement unit 503 and calculates the blood pressure value (systolic blood pressure value and diastolic blood pressure value) on the basis of this cuff pressure signal by applying a known algorithm by an oscillometric method. Upon calculating the blood pressure value, the control unit 213 performs control for storing the calculated blood pressure value in the storage unit 214. When measurement is completed, the control unit 213 opens the exhaust valve of the pump 221 via the pump drive circuit 220 and performs control for exhausting the air of the cuff structure 30. Note that the calculation of the blood pressure value is not limited to being performed in the depressurization process and may be performed in the pressurization process.

### Operational Example

### Communication Device

Next, an operation example of the communication device 170 will be described using FIG. 9. FIG. 9 is a flowchart illustrating an example of a processing procedure of the communication device 170.

### Activation

First, the user activates the communication device 170 and causes the activated communication device 170 to execute a third-party provision confirmation program. The control unit 413 of the communication device 170, according to the processing procedure below, stores in advance the personal information and the identification information of the user who has approved provision of the biological information and the personal information to a third party, accepts the identification information and the personal information of the blood pressure measurement device 100 that has entered the communication area of the communication device and, when these information is stored in the communication device 170, receives the identification information and the biological information from the blood pressure measurement device 100, and regards the user as having consented to providing, to a third party, the biological information and the personal information in association with each other. Furthermore, the communication device 170 can transmit a response signal indicating that the user consented to the blood pressure measurement device 100, and the user can recognize that the information is to be provided to a third party.

### Step S901

Step S901 is a procedure performed in advance, before executing the third-party provision confirmation program. To provide personal information and biological information to a third party from the user, it is necessary to clearly indicate to the individual (user) the content determination information indicating content of a reasonable and appropriate range considered necessary for the user to make a determination regarding consent, and then obtain consent from the user in advance. When this consent is obtained from the user, the personal information and identification information of the user who approved provision of biological information and personal information to a third party are stored in the storage unit 414 in advance.

### Step S902

In step S902, the control unit 413 functions as the identifier delivery unit 601, and broadcasts a packet including the identification information of the communication device 170 via the communication interface 417. The blood pressure measurement device 100 acquires the packet broadcast from the communication device 170, and the identifier delivery unit 601 decides whether or not the identification information included in the packet matches any one or more pieces of identification information stored in the storage unit 214 in advance. When the area confirmation unit 507 confirms that matching identification information exists in the storage unit 214, the blood pressure measurement device 100 approves the communication device 170 as a desired communication partner.

### Step S903

In step S903, the control unit 413 functions as the authentication unit 602, receives authentication from the blood pressure measurement device 100 and, performs authentication for the blood pressure measurement device 100 to perform encrypted communication with the communication device 170, and completes this authentication. Accordingly, subsequent encrypted communication between the blood pressure measurement device 100 and the communication device 170 is realized.

### Step S904

In step S904, the control unit 413 functions as the cross-checking unit 603, and decides whether or not the identification information of the blood pressure measurement device 100 serving as the communication partner and the personal information associated with this identification information are stored in the storage unit 414. When this identification information and personal information are stored in the storage unit 414, the communication device 170 transmits a signal indicating that the information is stored in the storage unit 414 to the blood pressure measurement device 100, and the flow proceeds to step S905. On the other hand, when these information are not stored in the storage unit 414, this blood pressure measurement device 100 decides that consent was not obtained from the user in advance, and the flow proceeds to step S908.

### Step S905

In step S905, the control unit 413 functions as the authentication unit 602 and, after the blood pressure measurement device 100 receives the signal indicating that the identification information of the blood pressure measurement device 100 serving as the communication partner and the personal information associated with this identification information are stored in the storage unit 414, the blood pressure measurement device 100 transmits the identification information of the blood pressure measurement device 100 and the biological information associated with this identification information to the communication device 170, and the communication device 170 acquires these information. The communication device 170 stores this identification information and biological information in the storage unit 414.

### Step S906

In step S906, the control unit 413 functions as the consent unit 604 and, when the identification information and the personal information received from the blood pressure measurement device 100 are stored in the storage unit 414, regards the user corresponding to the personal information associated with the identification information as having consented to providing, to a third party, the personal information of the user and the biological information of the user in association with each other. Then, the consent unit 604 transmits a response signal indicating that the user of the blood pressure measurement device 100 consented to the blood pressure measurement device 100 via the communication interface 417.

### Step S907

In step S907, the control unit 413 functions as the cross-checking unit 603, decides whether or not all items in the table shown in FIG. 6B stored in the storage unit 414 are filled, and, for example, proceeds to step S908 when all items are not filled, and terminates the program when all items are filled. Different from this, in order to update measured values, the flow may return to step S902 and wait for access from the blood pressure measurement device.

### Step S908

In step S908, the control unit 413 functions as the cross-checking unit 603 and, in order to transition to a state of receiving the identification information and the personal information from the another blood pressure measurement device, waits for authentication from the blood pressure measurement device storing the identification information included in the packet broadcast by the communication device 170.

### Actions and Effects

As described above, in the present embodiment, when a user consents to providing, to a third party, biological information and personal information via a specific communication device (communication device 170), and the communication device 170 stores the identification information and the personal information of the blood pressure measurement device 100 serving as the communication partner in steps S706 and S904 described above, the identification information of the blood pressure measurement device 100 and the corresponding biological information are transmitted to the communication device 170 in steps S707 and S905, making it possible to provide the biological information and the personal information of the user to a third party via a specific communication device. Thus, according to the present embodiment, it is possible to identify whether or not a user consents to third-party provision of personal information including biological information.

### Modified Example

While embodiments of the present invention have been described in detail above, the foregoing description is merely illustrative of the present invention in all respects. Of course, various modifications and variations can be made without departing from the scope of the present invention. For example, the following changes are possible. Note that, in the following, the same reference numerals are used for components similar to those of the above-described embodiment, and descriptions thereof will be omitted as appropriate. The following modified examples can be combined as appropriate.

1

### Hardware Configuration

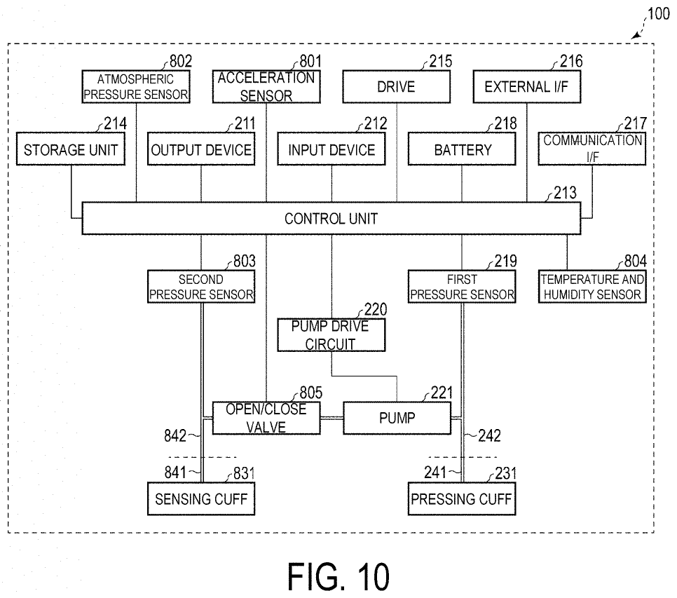

In the embodiment described above, the blood pressure measurement device 100 includes the output device 211, the input device 212, the control unit 213, the storage unit 214, the drive 215, the external interface 216, the communication interface 217, and the computer to which the battery 218 is electrically connected, as illustrated in FIG. 2. Nevertheless, other devices configured to perform various types of information processing may also be provided. For example, as illustrated in FIG. 10, the blood pressure measurement device 100 may further include an acceleration sensor 801, an atmospheric pressure sensor 802, a second pressure sensor 803, a temperature and humidity sensor 804, an open/close valve 805, and a sensing cuff 831.

The acceleration sensor 801 is a three-axis acceleration sensor. The acceleration sensor 801 outputs an acceleration signal indicating acceleration in three directions orthogonal to each other to the control unit 213.

The atmospheric pressure sensor 802 detects atmospheric pressure. The atmospheric pressure sensor 802 outputs the atmospheric pressure data to the control unit 213.

The temperature and humidity sensor 804 measures the ambient temperature and ambient humidity surrounding the blood pressure measurement device 100, and outputs the temperature and humidity data to the control unit 213.

Further, although not illustrated, the blood pressure measurement device 100 may include a GPS receiver.

The GPS receiver respectively receives GPS signals transmitted from a plurality of GPS satellites, and outputs the received GPS signals to the control unit 213. The control unit 213 calculates the current position information of the blood pressure measurement device 100, that is, the position of the subject (user) to which the blood pressure measurement device 100 is attached by performing a distance measurement calculation on the basis of each of the GPS signals described above.

Note that the blood pressure measurement device 100 need not necessarily include the distance measurement calculation function formed by the GPS receiver and the control unit 213. In this case, the blood pressure measurement device 100 obtains position information calculated by the communication device 170 from the communication device 170 via the communication interface 217.

Note that, in this case, the battery 218 supplies power to, for example, the output device 211, the control unit 213, the storage unit 214, the acceleration sensor 801, the atmospheric pressure sensor 802, the temperature and humidity sensor 804, the communication interface 217, the (first) pressure sensor 219, the second pressure sensor 803, the pump drive circuit 220, the pump 221, the open/close valve 805, and the GPS receiver (not illustrated).

The cuff structure 30 includes the curler 301, the pressing cuff 231, the back plate 303, and the sensing cuff 831.

The back plate 303 is disposed along the inner peripheral surface of the pressing cuff 231. The back plate 303 is band-like. The back plate 303 is constituted of, for example, a resin. The resin is, for example, polypropylene. The back plate 303 functions as a reinforcing plate. Thus, the back plate 303 can transmit the pressing force from the pressing cuff 231 across the entire area of the sensing cuff 831.

The back plate 303 is interposed between the pressing cuff 231 and the sensing cuff 831 and extends along in the Z direction. The sensing cuff 831 extends in the Z direction, coming into contact with the left wrist and crossing an artery passing portion of the left wrist. The belt 20, the curler 301, the pressing cuff 231, and the back plate 303 act as a pressing member capable of generating a pressing force toward the left wrist, and compress the left wrist by interposing the sensing cuff 831.

The open/close valve 805 is interposed in a second flow path formation member 842. The open/close valve 805 is, for example, a normally-open solenoid valve. The opening and closing of the open/close valve 805 is controlled on the basis of a control signal from the control unit 213. When the open/close valve 805 is in the open state, the pump 221 can supply fluid to the sensing cuff 831 through a flexible tube 841 and the second flow channel formation member 842 constituting the second flow path.

2

### Software Configuration

The blood pressure measurement device 100 according to the present embodiment may function as a computer further including an activity level measurement unit, a step counting unit, a sleep state measurement unit, and an environment (temperature and humidity) measurement unit. The storage unit 214 stores, for example, respective programs (an activity level measurement program, a step counting program, a sleep state measurement program, and an environment (temperature and humidity) measurement program), and expands the desired program into the RAM when the necessary program is to be executed. Then, the control unit 213 interprets and executes the program expanded into the RAM by the CPU to control each component.

The blood pressure measurement device 100 may record, for example, biological information of the activity level, the number of steps, and/or the sleep state, instead of or in addition to time series data of a blood pressure value.

The activity level measurement unit detects acceleration by the acceleration sensor 801 and calculates the activity level. The activity level measurement unit may use the acceleration signal to calculate not only the walking of the subject, but also the activity level of various activities such as household work or desk work. The activity level is, for example, an indicator associated with the activity of the subject, such as walking distance, calories consumption, or the amount of fat burned.

The step counting unit detects acceleration by the acceleration sensor 801 and atmospheric pressure by the atmospheric pressure sensor 802, and calculates a number of steps, a number of fast-paced steps, and a number of stairs climbed. The acceleration signal is used to calculate the walking of the subject. The step counting unit can use the atmospheric pressure data and the acceleration signal to calculate the number of steps, the number of fast-paced steps, the number of stairs climbed, and the like of the subject.

The sleep state measurement unit detects acceleration by the acceleration sensor 801 and detects the state of tosses and turns by the acceleration sensor, making it possible to estimate the sleep state.

The environment (temperature and humidity) measurement unit correlates environment data indicating the ambient temperature and the ambient humidity measured by the temperature and humidity sensor 804 with a measurement time of the temperature and humidity sensor 804, and stores the environment data in the storage unit 214. Temperature (change in temperature) is considered to be one factor that can cause blood pressure fluctuations in humans, for example. Therefore, the environment data is information that can be a factor in blood pressure fluctuations of the subject.

3

The blood pressure measurement device 100 may initiate blood pressure measurement (performing the operation of FIG. 8) when the operation unit 107 included in the input device 212 is pressed (turned on).

4

In the embodiment described above, the cuff structure 30 is used to measure the blood pressure value of the user by an oscillometric method. Nevertheless, when only the blood pressure value is measured, the method is not limited thereto. For example, a pressure pulse wave sensor that detects a pressure pulse wave per heartbeat may be provided to detect a pressure pulse wave of a radial artery passing through a target measurement site (left wrist, for example) to measure the blood pressure value (systolic blood pressure value and diastolic blood pressure value) (tonometry method). The pressure pulse wave sensor may detect a pulse wave of a radial artery passing through the target measurement site (left wrist, for example) as a change in impedance to measure the blood pressure value (impedance method). The pressure pulse wave sensor may include a light emitting element that irradiates light at the artery running through the corresponding portion of the target measurement site and a light receiving element that receives the reflected light (or transmitted light) of the light, the pulse wave of the artery being detected as a change in volume, thereby measuring the blood pressure value (photoelectric method).

Alternatively, the pressure pulse wave sensor may include a piezoelectric sensor abutting against the target measurement site, the strain due to the pressure of the artery running through the corresponding portion of the target measurement site being detected as a change in electric resistance, thereby measuring the blood pressure value (piezoelectric method). In another alternate, the pressure pulse wave sensor may include a transmission element that transmits an electromagnetic wave (transmission wave) to the artery running through the corresponding portion of the target measurement site and a reception element that receives the reflected wave of the electromagnetic wave, the change in distance between the artery and the sensors due to the pulse wave of the artery being detected as a phase shift between the transmission wave and the reflected wave, thereby detecting the blood pressure value (electromagnetic wave irradiation method). Note that as long as a physical quantity capable of calculating the blood pressure value can be observed, a method other than these may be applied.

5

The device of the present invention can also be realized by a computer and a program, and the program can be recorded on a recording medium (or storage medium) and provided through a network.

Further, the above-described devices and the device portions thereof can each be implemented by either a hardware configuration or a combined configuration of a hardware resource and software. As the software of the combined configuration, a program is used which is installed in a computer from a network or a computer-readable recording medium (or storage medium) in advance, and is executed by a processor of the computer, thereby realizing the functions of the respective devices by the computer.

Note that this invention is not limited to the embodiments described above, and can be embodied by modifying the components in an implementation stage without departing from the gist thereof. Further, various inventions can be formed by appropriate combinations of the plurality of components disclosed in the embodiments described above. For example, from among all components illustrated in the embodiments, several components may be deleted. Furthermore, components of different embodiments may be combined as appropriate.

Further, “and/or” means that any one or more items of the items connected and listed by the “and/or” statement. As a specific example, “x and/or y” means any element of the set of three elements {(x), (y), (x, y)}. As another specific example, “x, y, and/or z” means any element of the set of seven elements {(x), (y), (z), (x, y), (x, z), (y, z), (x, y, z)}.

6

Further, the whole or part of the embodiments described above can be described as, but not limited to, the following supplementary notes.

### Supplementary Note 1

A biological information measuring device configured to communicate with a communication device having a communication area of a specific range and measure information of a living body pertaining to a user, including

a hardware processor; and

a memory,

the hardware processor being configured to

transmit, when the biological information measuring device is detected as having entered the communication area, identification information unique to the biological information measuring device to the communication device, and

receive, when the communication device has stored in advance the identification information and personal information of the user associated with the identification information, a response signal from the communication device indicating that consent is regarded as having been obtained for providing, to a third party, the personal information and the information of the living body in association with each other, and

the memory including

a storage unit configured to store the information of the living body, the personal information, and the identification information.

### Supplementary Note 2

A communication device configured to communicate with a biological information measuring device configured to measure information of a living body pertaining to a user and having a communication area in a specific range, including

a hardware processor; and

a memory,

the memory including

a storage unit configured to store in advance identification information of at least one biological information measuring device and personal information of a user who uses the biological information measuring device in association with each other,

the hardware processor being configured to

receive identification information unique to the biological information measurement device and the information of the living body measured by the biological information measurement device when the user has consented to providing, to a third party, the information of the living body and the personal information of the user in association with each other and the biological information measuring device has entered the communication area, and

regard the user corresponding to personal information associated with the identification information as having consented to providing, to a third party, the personal information and the information of the living body in association with each other when the identification information thus received is stored in the storage unit.

### Supplementary Note 3

A communication device configured to communicate with a biological information measuring device configured to measure information of a living body pertaining to a user and having a communication area in a specific range, the communication device including

a hardware processor; and

a memory,

the hardware processor being configured to

receive identification information unique to the biological information measuring device and the information of the living body when the biological information measuring device has entered the communication area,

transmit, to the biological information measuring device, consent determination information indicating content necessary for determining whether or not to consent to providing, to a third party, the information of the living body in association with personal information associated with the identification information, and

regard the user as having consented to providing, to a third party, the personal information and the information of the living body in association with each other when a signal indicating that the user consented on the basis of the content indicated by the consent determination information is received from the biological information measuring device, and

the memory including

a storage unit configured to store the information of the living body, the personal information, and the identification information.

### Supplementary Note 4

A system including

a biological information measuring device configured to measure information of a living body pertaining to a user provided with a first hardware processor and a first memory; and

a communication device including a second hardware processor and a second memory, and having a communication area of a specific range,

the first hardware processor being configured to

present, to the user, consent determination information indicating content necessary for the user to determine whether or not to consent to providing, to a third party, the information of the living body and personal information of the user in association with each other,

receive, from the user, a signal indicating a determination result made by the user regarding consent on the basis of the content indicated by the consent determination information, and

transmit identification information unique to the biological information measuring device and the information of the living body to the communication device when the signal indicating the determination result indicates consent and the biological information measuring device has entered the communication area,

the first memory including

a storage unit configured to store the information of the living body, the personal information, and the identification information,

the second memory including

a storage unit configured to store in advance identification information of at least one biological information measuring device and personal information in association with each other, and

the second hardware processor being configured to

decide whether or not the identification information transmitted by the biological information measuring device is stored in the storage unit, and

when the identification information transmitted by the biological information measuring device is decided to be stored in the storage unit, regard the user included in personal information associated with the identification information as having consented to providing, to a third party, the personal information and the information of the living body in association with each other.

### Supplementary Note 5

A biological information measurement method including

transmitting by using at least one hardware processor, when a biological information measuring device is detected as having entered a communication area, identification information unique to the biological information measuring device to the communication device; and

receiving by using at least one hardware processor, when the identification information and personal information of a user associated with the identification information have been stored in advance by the communication device, a response signal from the communication device indicating that consent is regarded as having been obtained for providing, to a third party, the personal information and the information of the living body in association with each other.

### Supplementary Note 6

A communication method including

receiving by using at least one hardware processor, when a biological information measuring device has entered a communication area, identification information unique to the biological information measuring device and the information of the living body from the biological information measuring device; and

regarding by using at least one hardware processor, when a signal indicating that the user has consented on the basis of personal information associated with the identification information is received from the biological information measuring device, the user as having consented to providing, to a third party, the personal information and the information of the living body in association with each other.

### Supplementary Note 7

A communication method including

receiving by using at least one hardware processor, when a biological information measuring device has entered the communication area, identification information unique to the biological information measuring device and the information of the living body from the biological information measuring device;
transmitting by using at least once hardware processor, to the biological information measuring device, consent determination information indicating content necessary for determining whether or not to consent to providing, to a third party, the information of the living body in association with personal information associated with the identification information; and

regarding by using at least one hardware processor, when a signal indicating that the user consented on the basis of the content indicated by the consent determination information is received from the biological information measuring device, the user as having consented to providing to, a third party, the personal information and the information of the living body in association with each other.

### Supplementary Note 8

A method including

presenting by using at least one hardware processor, to a user, consent determination information indicating content necessary for the user to determine whether or not to consent to providing, to a third party, information of a living body pertaining to the user and personal information of the user in association with each other;

receiving by using at least one hardware processor, from the user, a signal indicating a determination result made by the user regarding consent on the basis of the content indicated by the consent determination information; and

transmitting by using at least one hardware processor, when the signal indicating the determination result indicates consent and the biological information measuring device has entered the communication area, identification information unique to the biological information measuring device and the information of the living body to the communication device.

### REFERENCE SIGNS LIST

10 . . . Body
10A . . . Case, 10B Glass, 10C . . . Rear lid
20 . . . Belt
30 . . . Cuff structure, 30a . . . First end, 30b . . . Second end
100 . . . Blood pressure measuring device
101 . . . Presentation unit
102 . . . Determination reception unit
103 . . . Authentication unit
104 . . . Transmission unit
105 . . . Reception unit
106 . . . Blood pressure measurement unit
107 . . . Operation unit
170 . . . Communication device
171 . . . Reception unit
172 . . . Storage unit
173 . . . Provision unit
174 . . . Transmission unit
175 . . . Authentication unit
190 . . . Server
201 . . . Belt portion
201a . . . Base portion, 201b . . . Tip end portion
202 . . . Belt portion, 202a . . . Base portion, 202b . . . Tip end portion, 202c . . . Small hole
203 . . . Buckle, 203A . . . Frame body, 203B . . . Buckle tongue, 203C . . . Connecting rod
204 . . . Belt holding portion
211 . . . Output device
212 . . . Input device
213 . . . Control unit
214 . . . Storage unit
215 . . . Drive
216 . . . External interface
217 . . . Communication interface
218 . . . Battery
219 . . . Pressure sensor
220 . . . Pump drive circuit
221 . . . Pump
231 . . . Pressing cuff
241 . . . Flexible tube
242 . . . Flow path formation member
301 . . . Curler, 303 Back plate
401 . . . Connecting rod, 402 . . . Connecting rod
411 . . . Output device
412 . . . Input device
413 . . . Control unit
414 . . . Storage unit
415 . . . Drive
416 . . . External interface
417 . . . Communication interface
418 . . . Battery
501 . . . Presentation unit
502 . . . Determination reception unit
503 . . . Blood pressure measurement unit
504 . . . Identification information decision unit
505 . . . Authentication unit
506 . . . Response reception unit
507 . . . Area confirmation unit
508 . . . Notification unit
601 . . . Identifier delivery unit
602 . . . Authentication unit
603 . . . Cross-checking unit
604 . . . Consent unit
801 . . . Acceleration sensor
802 . . . Atmospheric pressure sensor
803 . . . Pressure sensor
804 . . . Temperature and humidity sensor
805 . . . Open/close valve
831 . . . Sensing cuff
841 . . . Flexible tube
842 . . . Flow path formation member

1. A biological information measuring device having a function for communicating with a communication device having a communication area of a specific range and a function for measuring information of a living body pertaining to a user, the biological information measuring device comprising:
a processor; and
a memory, wherein
the processor is configured to transmit, when the biological information measuring device is detected as having entered the communication area, identification information unique to the biological information measuring device to the communication device, and
the processor is configured to receive, when the communication device has stored in advance the identification information and personal information of the user associated with the identification information, a response signal from the communication device indicating that consent is regarded as having been obtained for providing, to a third party, the personal information and the information of the living body in association with each other.

2. The biological information measuring device according to claim 1, wherein
the processor is configured to present, to the user, consent determination information indicating content necessary for the user to make a determination whether or not to consent to providing, to a third party, the information of the living body and personal information of the user corresponding to the living body in association with each other,
the processor is configured to receive, from the user, a signal indicating a determination result made by the user regarding consent on the basis of the content indicated by the consent determination information, and
the processor is configured to transmit, when the signal indicating the determination result indicates consent and the biological information measuring device is detected as having entered the communication area, the identification information and the information of the living body to the communication device.

3. The biological information measuring device according to claim 2, wherein
the processor is configured to present the consent determination information to the user when the biological information measuring device is detected as having entered the communication area, and
the processor is configured to receive a signal indicating the determination when the biological information measuring device is detected as having entered the communication area.

4. The biological information measuring device according to claim 2, wherein
the processor is configured to present the consent determination information to the user without the biological information measuring device detected as having entered the communication area, and
the processor is configured to receive a signal indicating the determination without the biological information measuring device detected as having entered the communication area.

5. The biological information measuring device according to claim 1, wherein
the memory is configured to store in advance authentication information of a user for performing encrypted communication,
the processor is configured to transmit the authentication information to the communication device and perform authentication for performing encrypted communication with the communication device, and
the processor is configured to initiate encrypted communication with the communication device when the authentication is completed.

6. A communication device configured to communicate with a biological information measuring device configured to measure information of a living body pertaining to a user and having a communication area in a specific range, the communication device comprising:
a processor; and
a memory, wherein
the processor is configured to receive identification information unique to the biological information measuring device and the information of the living body measured by the biological information measuring device from the biological information measuring device when the user consents to providing, to a third party, the information of the living body and personal information of the user, in association with each other, and the biological information measuring device has entered the communication area,
the memory is configured to store in advance identification information of at least one biological information measuring device and personal information of a user using the biological information measuring device in association with each other, and
the processor is configured to regard the user corresponding to personal information associated with the identification information as having consented to providing, to a third party, the personal information and the information of the living body in association with each other when the identification information being received is stored in the memory.

7. A communication device configured to communicate with a biological information measuring device configured to measure information of a living body pertaining to a user and having a communication area in a specific range, the communication device comprising:
a processor; and
a memory, wherein
the processor is configured to receive identification information unique to the biological information measuring device and the information of the living body from the biological information measuring device when the biological information measuring device has entered the communication area,
the processor is configured to transmit, to the biological information measuring device, consent determination information indicating content necessary for a user corresponding to the personal information to determine whether or not to consent to providing, to a third party, the information of the living body in association with personal information associated with the identification information, and
the processor is configured to regard the user as having consented to providing, to a third party, the personal information and the information of the living body in association with each other when a signal indicating that the user consented on the basis of the content indicated by the consent determination information is received from the biological information measuring device.

8. The communication device according to claim 6, further comprising:
a memory configured to store in advance authentication information of a user for performing encrypted communication, wherein
the processor is configured to transmit the authentication information to the biological information measuring device and perform authentication for performing encrypted communication with the biological information measuring device, and
the processor is configured to initiate encrypted communication with the biological information measuring device when the authentication is completed.

9. The communication device according to claim 8,
wherein the processor is configured to decide whether or not identification information obtained when the biological information measuring device logs into the communication device using the authentication information differs from the identification information associated with the personal information, and
wherein the processor is configured to notify, when decision with the identification information obtained differing from the identification information associated with the personal information is made, information indicating the decision to a user corresponding to the personal information.

10. The communication device according to claim 8,
wherein the processor is configured to decide whether or not login information obtained when the biological information measuring device logs into the communication device using the authentication information differs from login information of the past included in login history, and
wherein the processor is configured to notify, when decision with the login information obtained differing from the login information included in the login history is made, information indicating the decision to a user corresponding to the personal information.

11. A system comprising:
a biological information measuring device configured to measure information of a living body pertaining to a user; and
a communication device having a communication area of a specific range, wherein
the biological information measuring device includes:
a first processor; and
a first memory, wherein
the first processor is configured to present, to the user, consent determination information indicating content necessary for the user to determine whether or not to consent to providing, to a third party, the information of the living body and personal information of the user in association with each other,
the first processor is configured to receive, from the user, a signal indicating a determination result made by the user regarding consent on the basis of the content indicated by the consent determination information, and
the first processor is configured to transmit identification information unique to the biological information measuring device and the information of the living body to the communication device when the signal indicating the determination result indicates consent and the biological information measuring device has entered the communication area, and
the communication device includes:
a second processor; and
a second memory, wherein
the second memory is a storage unit configured to store in advance identification information of at least one biological information measuring device and personal information of a user using the biological information measuring device in association with each other,
the second processor is configured to decide whether or not the identification information transmitted by the biological information measuring device is stored in the second memory, and
the second processor is configured to regard the user as having consented to providing, to a third party, personal information associated with the identification information and the information of the living body in association with each other when the identification information transmitted by the biological information measuring device is decided to be stored in the second memory.

12. A biological information measurement method performed by a biological information measuring device configured to communicate with a communication device having a communication area of a specific range and measure information of a living body pertaining to a user, the biological information measurement method comprising:
transmitting, when the biological information measuring device is detected as having entered the communication area, identification information unique to the biological information measuring device to the communication device; and
receiving, when the communication device has stored in advance the identification information and personal information of the user associated with the identification information, a response signal from the communication device indicating that consent is regarded as having been obtained for providing, to a third party, the personal information and the information of the living body in association with each other.

13. A communication method performed by a communication device configured to communicate with a biological information measuring device configured to measure information of a living body pertaining to a user and having a communication area in a specific range, the communication method comprising:
receiving identification information unique to the biological information measuring device and the information of the living body measured by the biological information measuring device when the user consents to providing, to a third party, the information of the living body and personal information of the user in association with each other and the biological information measuring device has entered the communication area; and
regarding, when the identification information being received and personal information associated with the identification are stored in advance, the user as having consented to providing, to a third party, the personal information and the information of the living body in association with each other.

14. A non-transitory recording medium storing a program for causing the processor included in the biological information measuring device according to claim 1 to transmit and receive.

15. A non-transitory recording medium storing a program for causing the processor included in the communication device according to claim 6 to receive, store and regard.

16. The biological information measuring device according to claim 2, wherein
the memory is configured to store in advance authentication information of a user for performing encrypted communication,
the processor is configured to transmit the authentication information to the communication device and perform authentication for performing encrypted communication with the communication device, and
the processor is configured to initiate encrypted communication with the communication device when the authentication is completed.

17. The biological information measuring device according to claim 3, wherein
the memory is configured to store in advance authentication information of a user for performing encrypted communication,
the processor is configured to transmit the authentication information to the communication device and perform authentication for performing encrypted communication with the communication device, and
the processor is configured to initiate encrypted communication with the communication device when the authentication is completed.

18. The biological information measuring device according to claim 4, wherein
the memory is configured to store in advance authentication information of a user for performing encrypted communication,
the processor is configured to transmit the authentication information to the communication device and perform authentication for performing encrypted communication with the communication device, and
the processor is configured to initiate encrypted communication with the communication device when the authentication is completed.

19. The communication device according to claim 7, wherein
the memory is configured to store in advance authentication information of a user for performing encrypted communication,
the processor is configured to transmit the authentication information to the biological information measuring device and perform authentication for performing encrypted communication with the biological information measuring device, and
the processor is configured to initiate encrypted communication with the biological information measuring device when the authentication is completed.

20. A non-transitory recording medium storing a program for causing the processor included in the biological information measuring device according to claim 2 to transmit, receive, and present.
